SEQUENCE LISTING (60 sequences)

SEQ ID NO: 1 - DNA, 47 bp, synthetic construct
  source 1..47: mol_type = other DNA
  modified_base 4: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 31: mod_base = OTHER; note = 5-tryptamino-uracil
  modified_base 36: mod_base = OTHER; note = 5-tryptamino-uracil
  modified_base 45: mod_base = OTHER; note = 5-tryptamino-uracil
ccatacagattaacaaaaacattacttttttaaagtggacaaaatcc

SEQ ID NO: 2 - DNA, 47 bp, synthetic construct
  source 1..47: mol_type = other DNA
  modified_base 9: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 18: mod_base = OTHER; note = 5-tryptamino-uracil
  modified_base 20: mod_base = OTHER; note = 5-tryptamino-uracil
  modified_base 31: mod_base = OTHER; note = 5-tryptamino-uracil
  modified_base 39: mod_base = OTHER; note = 5-tryptamino-uracil
ccagaacgttgtagccatatgcgcacgacgtaacgacctaagcaacc

SEQ ID NO: 3 - DNA, 47 bp, synthetic construct
  source 1..47: mol_type = other DNA
  modified_base 4: mod_base = OTHER; note = 5-tryptamino-uracil
  modified_base 5: mod_base = OTHER; note = 5-tryptamino-uracil
  modified_base 7: mod_base = OTHER; note = 5-tryptamino-uracil
  modified_base 13: mod_base = OTHER; note = 5-tryptamino-uracil
  modified_base 15: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 35: mod_base = OTHER; note = 5-tryptamino-uracil
  modified_base 40: mod_base = OTHER; note = 5-tryptamino-uracil
ccgttgtagccatatgcggaacaagaattagggatcgcatcgcaacc

SEQ ID NO: 4 - DNA, 47 bp, synthetic construct
  source 1..47: mol_type = other DNA
  modified_base 3: mod_base = OTHER; note = 5-tryptamino-uracil
  modified_base 13: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 30: mod_base = OTHER; note = 5-tryptamino-uracil
  modified_base 34: mod_base = OTHER; note = 5-tryptamino-uracil
  modified_base 38: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil
cctaacccaaaatcccacatgatattgtgtccatacatccatcatcc

SEQ ID NO: 5 - DNA, 47 bp, synthetic construct
  source 1..47: mol_type = other DNA
  modified_base 3: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 32: mod_base = OTHER; note = 5-tryptamino-uracil
  modified_base 43: mod_base = OTHER; note = 5-tryptamino-uracil
cctaagaaacggaatttattttctcttaaagtggacgagacgtgacc

SEQ ID NO: 6 - DNA, 47 bp, synthetic construct
  source 1..47: mol_type = other DNA
  modified_base 12: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 37: mod_base = OTHER; note = 5-tryptamino-uracil
  modified_base 41: mod_base = OTHER; note = 5-tryptamino-uracil
  modified_base 44: mod_base = OTHER; note = 5-tryptamino-uracil
ccacgaaggaatacacttgtttttatgttagaggactggatgatccc

SEQ ID NO: 7 - DNA, 47 bp, synthetic construct
  source 1..47: mol_type = other DNA
  modified_base 3: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 15: mod_base = OTHER; note = 5-tryptamino-uracil
  modified_base 18: mod_base = OTHER; note = 5-tryptamino-uracil
  modified_base 21: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 38: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil
cctagagggatgtgtagtgctcggagcatccctgaaatgcatcatcc

SEQ ID NO: 8 - DNA, 47 bp, synthetic construct
  source 1..47: mol_type = other DNA
  modified_base 3: mod_base = OTHER; note = 5-tryptamino-uracil
  modified_base 14: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 31: mod_base = OTHER; note = 5-tryptamino-uracil
  modified_base 39: mod_base = OTHER; note = 5-tryptamino-uracil
cctaaaacgaagatgactttattttttcagtggacggatacggagcc

SEQ ID NO: 9 - DNA, 47 bp, synthetic construct
  source 1..47: mol_type = other DNA
  modified_base 4: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 12: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 24: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 32: mod_base = OTHER; note = 5-tryptamino-uracil
  modified_base 40: mod_base = OTHER; note = 5-tryptamino-uracil
  modified_base 43: mod_base = OTHER; note = 5-tryptamino-uracil
ccgtacacatttggctttttaaatgatttcataagggactgataacc

SEQ ID NO: 10 - DNA, 47 bp, synthetic construct
  source 1..47: mol_type = other DNA
  modified_base 4: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 32: mod_base = OTHER; note = 5-tryptamino-uracil
  modified_base 36: mod_base = OTHER; note = 5-tryptamino-uracil
  modified_base 38: mod_base = OTHER; note = 5-tryptamino-uracil
  modified_base 40: mod_base = OTHER; note = 5-tryptamino-uracil
ccataaagaccgccgggacgggcgactgttgtagctatatgcagccc

SEQ ID NO: 11 - DNA, 47 bp, synthetic construct
  source 1..47: mol_type = other DNA
  modified_base 11: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 40: mod_base = OTHER; note = 5-tryptamino-uracil
cccgcccaactaattttattcttctaagtggatgaaagatccgcacc

SEQ ID NO: 12 - DNA, 47 bp, synthetic construct
  source 1..47: mol_type = other DNA
  modified_base 12: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 38: mod_base = OTHER; note = 5-tryptamino-uracil
  modified_base 39: mod_base = OTHER; note = 5-tryptamino-uracil
  modified_base 42: mod_base = OTHER; note = 5-tryptamino-uracil
ccaacgaacgatcattaggttgaagctatatgccaggttcatgaccc

SEQ ID NO: 13 - DNA, 47 bp, synthetic construct
  source 1..47: mol_type = other DNA
  modified_base 14: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 31: mod_base = OTHER; note = 5-tryptamino-uracil
  modified_base 38: mod_base = OTHER; note = 5-tryptamino-uracil
  modified_base 45: mod_base = OTHER; note = 5-tryptamino-uracil
cccaaaaacagcattctttcttttataaagtggaccatcaaagctcc

SEQ ID NO: 14 - DNA, 47 bp, synthetic construct
  source 1..47: mol_type = other DNA
  modified_base 7: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 30: mod_base = OTHER; note = 5-tryptamino-uracil
  modified_base 35: mod_base = OTHER; note = 5-tryptamino-uracil
  modified_base 36: mod_base = OTHER; note = 5-tryptamino-uracil
  modified_base 37: mod_base = OTHER; note = 5-tryptamino-uracil
ccaccatcatataaaattttgatttaaagtggactttacaggcaacc

SEQ ID NO: 15 - DNA, 47 bp, synthetic construct
  source 1..47: mol_type = other DNA
  modified_base 12: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 36: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil
ccaagacgacatgatattttgcttatatttgaaagtggacatcatcc

SEQ ID NO: 16 - DNA, 47 bp, synthetic construct
  source 1..47: mol_type = other DNA
  modified_base 3: mod_base = OTHER; note = 5-tryptamino-uracil
  modified_base 5: mod_base = OTHER; note = 5-tryptamino-uracil
  modified_base 6: mod_base = OTHER; note = 5-tryptamino-uracil
  modified_base 8: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 12: mod_base = OTHER; note = 5-tryptamino-uracil
  modified_base 13: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 20: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
  modified_base 43: mod_base = OTHER; note = 5-tryptamino-uracil
  modified_base 45: mod_base = OTHER; note = 5-tryptamino-uracil
cctgttgtgttttttaaagtggggtgaataagcaaaaactcttatcc

SEQ ID NO: 17 - DNA, 47 bp, synthetic construct
  source 1..47: mol_type = other DNA
  modified_base 10: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 19: mod_base = OTHER; note = 5-tryptamino-uracil
  modified_base 21: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 31: mod_base = OTHER; note = 5-tryptamino-uracil
  modified_base 36: mod_base = OTHER; note = 5-tryptamino-uracil
  modified_base 39: mod_base = OTHER; note = 5-tryptamino-uracil
cccaaaccgttgtagcaatatgcgccttcctgccctaatagaccacc

SEQ ID NO: 18 - DNA, 47 bp, synthetic construct
  source 1..47: mol_type = other DNA
  modified_base 3: mod_base = OTHER; note = 5-tryptamino-uracil
  modified_base 4: mod_base = OTHER; note = 5-tryptamino-uracil
  modified_base 8: mod_base = OTHER; note = 5-tryptamino-uracil
  modified_base 12: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 31: mod_base = OTHER; note = 5-tryptamino-uracil
  modified_base 36: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
ccttaagtacgtcaattgatcgttttttagtggactgactctaaacc

SEQ ID NO: 19 - DNA, 47 bp, synthetic construct
  source 1..47: mol_type = other DNA
  modified_base 12: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 30: mod_base = OTHER; note = 5-tryptamino-uracil
  modified_base 39: mod_base = OTHER; note = 5-tryptamino-uracil
  modified_base 41: mod_base = OTHER; note = 5-tryptamino-uracil
  modified_base 45: mod_base = OTHER; note = 5-tryptamino-uracil
ccaaacgacagtaaacagttacttttttataaggacgatatcgatcc

SEQ ID NO: 20 - DNA, 47 bp, synthetic construct
  source 1..47: mol_type = other DNA
  modified_base 3: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 41: mod_base = OTHER; note = 5-tryptamino-uracil
  modified_base 43: mod_base = OTHER; note = 5-tryptamino-uracil
  modified_base 45: mod_base = OTHER; note = 5-tryptamino-uracil
cctaagcagtatgaacctttatttttatggggcgagacgatctgtcc

SEQ ID NO: 21 - DNA, 47 bp, synthetic construct; identical to SEQ ID NO: 1
  source 1..47: mol_type = other DNA
  modified_base 4: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 31: mod_base = OTHER; note = 5-tryptamino-uracil
  modified_base 36: mod_base = OTHER; note = 5-tryptamino-uracil
  modified_base 45: mod_base = OTHER; note = 5-tryptamino-uracil

SEQ ID NO: 22 - DNA, 47 bp, synthetic construct; identical to SEQ ID NO: 2
  source 1..47: mol_type = other DNA
  modified_base 9: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 18: mod_base = OTHER; note = 5-tryptamino-uracil
  modified_base 20: mod_base = OTHER; note = 5-tryptamino-uracil
  modified_base 31: mod_base = OTHER; note = 5-tryptamino-uracil
  modified_base 39: mod_base = OTHER; note = 5-tryptamino-uracil

SEQ ID NO: 23 - DNA, 47 bp, synthetic construct; identical to SEQ ID NO: 3
  source 1..47: mol_type = other DNA
  modified_base 4: mod_base = OTHER; note = 5-tryptamino-uracil
  modified_base 5: mod_base = OTHER; note = 5-tryptamino-uracil
  modified_base 7: mod_base = OTHER; note = 5-tryptamino-uracil
  modified_base 13: mod_base = OTHER; note = 5-tryptamino-uracil
  modified_base 15: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 35: mod_base = OTHER; note = 5-tryptamino-uracil
  modified_base 40: mod_base = OTHER; note = 5-tryptamino-uracil

SEQ ID NO: 24 - DNA, 47 bp, synthetic construct; identical to SEQ ID NO: 12
  source 1..47: mol_type = other DNA
  modified_base 12: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 38: mod_base = OTHER; note = 5-tryptamino-uracil
  modified_base 39: mod_base = OTHER; note = 5-tryptamino-uracil
  modified_base 42: mod_base = OTHER; note = 5-tryptamino-uracil

SEQ ID NO: 25 - DNA, 47 bp, synthetic construct; identical to SEQ ID NO: 4
  source 1..47: mol_type = other DNA
  modified_base 3: mod_base = OTHER; note = 5-tryptamino-uracil
  modified_base 13: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 30: mod_base = OTHER; note = 5-tryptamino-uracil
  modified_base 34: mod_base = OTHER; note = 5-tryptamino-uracil
  modified_base 38: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil

SEQ ID NO: 26 - DNA, 47 bp, synthetic construct; identical to SEQ ID NO: 10
  source 1..47: mol_type = other DNA
  modified_base 4: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 32: mod_base = OTHER; note = 5-tryptamino-uracil
  modified_base 36: mod_base = OTHER; note = 5-tryptamino-uracil
  modified_base 38: mod_base = OTHER; note = 5-tryptamino-uracil
  modified_base 40: mod_base = OTHER; note = 5-tryptamino-uracil

SEQ ID NO: 27 - DNA, 47 bp, synthetic construct; identical to SEQ ID NO: 8
  source 1..47: mol_type = other DNA
  modified_base 3: mod_base = OTHER; note = 5-tryptamino-uracil
  modified_base 14: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 31: mod_base = OTHER; note = 5-tryptamino-uracil
  modified_base 39: mod_base = OTHER; note = 5-tryptamino-uracil

SEQ ID NO: 28 - DNA, 47 bp, synthetic construct
  source 1..47: mol_type = other DNA
  modified_base 3: mod_base = OTHER; note = 5-tryptamino-uracil
  modified_base 6: mod_base = OTHER; note = 5-tryptamino-uracil
  modified_base 7: mod_base = OTHER; note = 5-tryptamino-uracil
  modified_base 9: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 36: mod_base = OTHER; note = 5-tryptamino-uracil
cctggttgtagcaatatgccccggagtatcggtcgtccaaaagagcc

SEQ ID NO: 29 - DNA, 47 bp, synthetic construct; identical to SEQ ID NO: 7
  source 1..47: mol_type = other DNA
  modified_base 3: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 15: mod_base = OTHER; note = 5-tryptamino-uracil
  modified_base 18: mod_base = OTHER; note = 5-tryptamino-uracil
  modified_base 21: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 38: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil

SEQ ID NO: 30 - DNA, 47 bp, synthetic construct
  source 1..47: mol_type = other DNA
  modified_base 14: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 37: mod_base = OTHER; note = 5-tryptamino-uracil
  modified_base 45: mod_base = OTHER; note = 5-tryptamino-uracil
ccccgagagaaggttgtagcaatatgcccgaggggatccgaacctcc

SEQ ID NO: 31 - DNA, 47 bp, synthetic construct; identical to SEQ ID NO: 15
  source 1..47: mol_type = other DNA
  modified_base 12: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 36: mod_base = OTHER; note = 5-tryptamino-uracil
  modified_base 42: mod_base = OTHER; note = 5-tryptamino-uracil
  modified_base 45: mod_base = OTHER; note = 5-tryptamino-uracil

SEQ ID NO: 32 - DNA, 47 bp, synthetic construct; identical to SEQ ID NO: 13
  source 1..47: mol_type = other DNA
  modified_base 14: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 31: mod_base = OTHER; note = 5-tryptamino-uracil
  modified_base 38: mod_base = OTHER; note = 5-tryptamino-uracil
  modified_base 45: mod_base = OTHER; note = 5-tryptamino-uracil

SEQ ID NO: 33 - DNA, 47 bp, synthetic construct; identical to SEQ ID NO: 5
  source 1..47: mol_type = other DNA
  modified_base 3: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 32: mod_base = OTHER; note = 5-tryptamino-uracil
  modified_base 43: mod_base = OTHER; note = 5-tryptamino-uracil

SEQ ID NO: 34 - DNA, 47 bp, synthetic construct
  source 1..47: mol_type = other DNA
  modified_base 15: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 32: mod_base = OTHER; note = 5-tryptamino-uracil
  modified_base 36: mod_base = OTHER; note = 5-tryptamino-uracil
  modified_base 44: mod_base = OTHER; note = 5-tryptamino-uracil
cccaggacaaaagataccagaagatttttcttaagtggacggatacc

SEQ ID NO: 35 - DNA, 47 bp, synthetic construct; identical to SEQ ID NO: 11
  source 1..47: mol_type = other DNA
  modified_base 11: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 40: mod_base = OTHER; note = 5-tryptamino-uracil

SEQ ID NO: 36 - DNA, 47 bp, synthetic construct; identical to SEQ ID NO: 16
  source 1..47: mol_type = other DNA
  modified_base 3: mod_base = OTHER; note = 5-tryptamino-uracil
  modified_base 5: mod_base = OTHER; note = 5-tryptamino-uracil
  modified_base 6: mod_base = OTHER; note = 5-tryptamino-uracil
  modified_base 8: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 12: mod_base = OTHER; note = 5-tryptamino-uracil
  modified_base 13: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 20: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
  modified_base 43: mod_base = OTHER; note = 5-tryptamino-uracil
  modified_base 45: mod_base = OTHER; note = 5-tryptamino-uracil

SEQ ID NO: 37 - DNA, 47 bp, synthetic construct
  source 1..47: mol_type = other DNA
  modified_base 6: mod_base = OTHER; note = 5-tryptamino-uracil
  modified_base 12: mod_base = OTHER; note = 5-tryptamino-uracil
  modified_base 14: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 27: mod_base = OTHER; note = 5-tryptamino-uracil
  modified_base 30: mod_base = OTHER; note = 5-tryptamino-uracil
  modified_base 33: mod_base = OTHER; note = 5-tryptamino-uracil
  modified_base 39: mod_base = OTHER; note = 5-tryptamino-uracil
  modified_base 41: mod_base = OTHER; note = 5-tryptamino-uracil
ccccataagcgtgtctgcgtcattactagtactacacctgtcagccc

SEQ ID NO: 38 - DNA, 47 bp, synthetic construct
  source 1..47: mol_type = other DNA
  modified_base 4: mod_base = OTHER; note = 5-tryptamino-uracil
  modified_base 13: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 32: mod_base = OTHER; note = 5-tryptamino-uracil
  modified_base 34: mod_base = OTHER; note = 5-tryptamino-uracil
  modified_base 39: mod_base = OTHER; note = 5-tryptamino-uracil
ccgtcaacccaatcaacattttgtgttttattgtggactgacacacc

SEQ ID NO: 39 - DNA, 47 bp, synthetic construct
  source 1..47: mol_type = other DNA
  modified_base 3: mod_base = OTHER; note = 5-tryptamino-uracil
  modified_base 6: mod_base = OTHER; note = 5-tryptamino-uracil
  modified_base 9: mod_base = OTHER; note = 5-tryptamino-uracil
  modified_base 12: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 33: mod_base = OTHER; note = 5-tryptamino-uracil
  modified_base 37: mod_base = OTHER; note = 5-tryptamino-uracil
  modified_base 41: mod_base = OTHER; note = 5-tryptamino-uracil
  modified_base 43: mod_base = OTHER; note = 5-tryptamino-uracil
  modified_base 45: mod_base = OTHER; note = 5-tryptamino-uracil
cctaatgataatcaaaacagcttgtagctatatggctcagtctatcc

SEQ ID NO: 40 - DNA, 47 bp, synthetic construct
  source 1..47: mol_type = other DNA
  modified_base 3: mod_base = OTHER; note = 5-tryptamino-uracil
  modified_base 4: mod_base = OTHER; note = 5-tryptamino-uracil
  modified_base 6: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 16: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 29: mod_base = OTHER; note = 5-tryptamino-uracil
  modified_base 32: mod_base = OTHER; note = 5-tryptamino-uracil
  modified_base 35: mod_base = OTHER; note = 5-tryptamino-uracil
  modified_base 40: mod_base = OTHER; note = 5-tryptamino-uracil
  modified_base 44: mod_base = OTHER; note = 5-tryptamino-uracil
ccttgtccaatatactcattactcgtactagtcctcagctgactgcc

SEQ ID NO: 41 - DNA, 47 bp, synthetic construct
  source 1..47: mol_type = other DNA
  modified_base 4: mod_base = OTHER; note = 5-tryptamino-uracil
  modified_base 5: mod_base = OTHER; note = 5-tryptamino-uracil
  modified_base 6: mod_base = OTHER; note = 5-tryptamino-uracil
  modified_base 7: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 26: mod_base = OTHER; note = 5-tryptamino-uracil
  modified_base 30: mod_base = OTHER; note = 5-tryptamino-uracil
  modified_base 32: mod_base = OTHER; note = 5-tryptamino-uracil
  modified_base 37: mod_base = OTHER; note = 5-tryptamino-uracil
  modified_base 40: mod_base = OTHER; note = 5-tryptamino-uracil
  modified_base 41: mod_base = OTHER; note = 5-tryptamino-uracil
  modified_base 44: mod_base = OTHER; note = 5-tryptamino-uracil
  modified_base 45: mod_base = OTHER; note = 5-tryptamino-uracil
ccattttactctcatatacgaaaggtacctatagaatcattagttcc

SEQ ID NO: 42 - DNA, 46 bp, synthetic construct
  source 1..46: mol_type = other DNA
  modified_base 4: mod_base = OTHER; note = 5-tryptamino-uracil
  modified_base 7: mod_base = OTHER; note = 5-tryptamino-uracil
  modified_base 8: mod_base = OTHER; note = 5-tryptamino-uracil
  modified_base 9: mod_base = OTHER; note = 5-tryptamino-uracil
  modified_base 14: mod_base = OTHER; note = 5-tryptamino-uracil
  modified_base 18: mod_base = OTHER; note = 5-tryptamino-uracil
  modified_base 21: mod_base = OTHER; note = 5-tryptamino-uracil
  modified_base 24: mod_base = OTHER; note = 5-tryptamino-uracil
  modified_base 28: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 36: mod_base = OTHER; note = 5-tryptamino-uracil
  modified_base 39: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
  modified_base 44: mod_base = OTHER; note = 5-tryptamino-uracil
ccctgctttggagtggatactaatcgatttaggaatgcttatctcc

SEQ ID NO: 43 - DNA, 47 bp, synthetic construct
  source 1..47: mol_type = other DNA
  modified_base 8: mod_base = OTHER; note = 5-tryptamino-uracil
  modified_base 9: mod_base = OTHER; note = 5-tryptamino-uracil
  modified_base 11: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 23: mod_base = OTHER; note = 5-tryptamino-uracil
  modified_base 30: mod_base = OTHER; note = 5-tryptamino-uracil
  modified_base 31: mod_base = OTHER; note = 5-tryptamino-uracil
  modified_base 32: mod_base = OTHER; note = 5-tryptamino-uracil
  modified_base 35: mod_base = OTHER; note = 5-tryptamino-uracil
  modified_base 37: mod_base = OTHER; note = 5-tryptamino-uracil
  modified_base 41: mod_base = OTHER; note = 5-tryptamino-uracil
  modified_base 43: mod_base = OTHER; note = 5-tryptamino-uracil
ccaaacattctaatttgaattgtgaagcatttcctctccgtgtcacc

SEQ ID NO: 44 - DNA, 47 bp, synthetic construct
  source 1..47: mol_type = other DNA
  modified_base 3: mod_base = OTHER; note = 5-tryptamino-uracil
  modified_base 7: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 21: mod_base = OTHER; note = 5-tryptamino-uracil
  modified_base 22: mod_base = OTHER; note = 5-tryptamino-uracil
  modified_base 25: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 32: mod_base = OTHER; note = 5-tryptamino-uracil
  modified_base 33: mod_base = OTHER; note = 5-tryptamino-uracil
  modified_base 35: mod_base = OTHER; note = 5-tryptamino-uracil
  modified_base 41: mod_base = OTHER; note = 5-tryptamino-uracil
  modified_base 42: mod_base = OTHER; note = 5-tryptamino-uracil
cctcaatactatcagaacgattgatatgtatttataacaattaaacc

SEQ ID NO: 45 - DNA, 47 bp, synthetic construct
  source 1..47: mol_type = other DNA
  modified_base 3: mod_base = OTHER; note = 5-tryptamino-uracil
  modified_base 6: mod_base = OTHER; note = 5-tryptamino-uracil
  modified_base 9: mod_base = OTHER; note = 5-tryptamino-uracil
  modified_base 15: mod_base = OTHER; note = 5-tryptamino-uracil
  modified_base 16: mod_base = OTHER; note = 5-tryptamino-uracil
  modified_base 18: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 30: mod_base = OTHER; note = 5-tryptamino-uracil
  modified_base 34: mod_base = OTHER; note = 5-tryptamino-uracil
  modified_base 38: mod_base = OTHER; note = 5-tryptamino-uracil
  modified_base 41: mod_base = OTHER; note = 5-tryptamino-uracil
  modified_base 43: mod_base = OTHER; note = 5-tryptamino-uracil
  modified_base 44: mod_base = OTHER; note = 5-tryptamino-uracil
cctactggtgggaattctagggtacttaataagtccctcatattgcc

SEQ ID NO: 46 - DNA, 47 bp, synthetic construct
  source 1..47: mol_type = other DNA
  modified_base 4: mod_base = OTHER; note = 5-tryptamino-uracil
  modified_base 7: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 14: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 29: mod_base = OTHER; note = 5-tryptamino-uracil
  modified_base 36: mod_base = OTHER; note = 5-tryptamino-uracil
  modified_base 38: mod_base = OTHER; note = 5-tryptamino-uracil
  modified_base 45: mod_base = OTHER; note = 5-tryptamino-uracil
ccatactaggtgttgtgcattactttaatagcgcctatacaaagtcc

SEQ ID NO: 47 - DNA, 47 bp, synthetic construct
  source 1..47: mol_type = other DNA
  modified_base 5: mod_base = OTHER; note = 5-tryptamino-uracil
  modified_base 8: mod_base = OTHER; note = 5-tryptamino-uracil
  modified_base 9: mod_base = OTHER; note = 5-tryptamino-uracil
  modified_base 10: mod_base = OTHER; note = 5-tryptamino-uracil
  modified_base 11: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 31: mod_base = OTHER; note = 5-tryptamino-uracil
  modified_base 33: mod_base = OTHER; note = 5-tryptamino-uracil
  modified_base 34: mod_base = OTHER; note = 5-tryptamino-uracil
  modified_base 35: mod_base = OTHER; note = 5-tryptamino-uracil
  modified_base 42: mod_base = OTHER; note = 5-tryptamino-uracil
ccactacttttaaaaaggcacatagtgacgtatttaaacagtcgacc

SEQ ID NO: 48 - DNA, 47 bp, synthetic construct
  source 1..47: mol_type = other DNA
  modified_base 4: mod_base = OTHER; note = 5-tryptamino-uracil
  modified_base 5: mod_base = OTHER; note = 5-tryptamino-uracil
  modified_base 10: mod_base = OTHER; note = 5-tryptamino-uracil
  modified_base 14: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 20: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 32: mod_base = OTHER; note = 5-tryptamino-uracil
  modified_base 36: mod_base = OTHER; note = 5-tryptamino-uracil
  modified_base 37: mod_base = OTHER; note = 5-tryptamino-uracil
  modified_base 39: mod_base = OTHER; note = 5-tryptamino-uracil
  modified_base 40: mod_base = OTHER; note = 5-tryptamino-uracil
  modified_base 43: mod_base = OTHER; note = 5-tryptamino-uracil
ccattaccgtcaatattgatgaatgtcatattgacttgttggtcgcc

SEQ ID NO: 49 - DNA, 47 bp, synthetic construct
  source 1..47: mol_type = other DNA
  modified_base 4: mod_base = OTHER; note = 5-tryptamino-uracil
  modified_base 5: mod_base = OTHER; note = 5-tryptamino-uracil
  modified_base 8: mod_base = OTHER; note = 5-tryptamino-uracil
  modified_base 11: mod_base = OTHER; note = 5-tryptamino-uracil
  modified_base 17: mod_base = OTHER; note = 5-tryptamino-uracil
  modified_base 20: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 28: mod_base = OTHER; note = 5-tryptamino-uracil
  modified_base 32: mod_base = OTHER; note = 5-tryptamino-uracil
  modified_base 33: mod_base = OTHER; note = 5-tryptamino-uracil
  modified_base 34: mod_base = OTHER; note = 5-tryptamino-uracil
  modified_base 42: mod_base = OTHER; note = 5-tryptamino-uracil
  modified_base 44: mod_base = OTHER; note = 5-tryptamino-uracil
ccgttaataatcccgatactattttagtacatttggccaaatgtacc

SEQ ID NO: 50 - DNA, 47 bp, synthetic construct
  source 1..47: mol_type = other DNA
  modified_base 7: mod_base = OTHER; note = 5-tryptamino-uracil
  modified_base 12: mod_base = OTHER; note = 5-tryptamino-uracil
  modified_base 14: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 27: mod_base = OTHER; note = 5-tryptamino-uracil
  modified_base 34: mod_base = OTHER; note = 5-tryptamino-uracil
  modified_base 36: mod_base = OTHER; note = 5-tryptamino-uracil
  modified_base 38: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
ccaaaataacatctgccctttacaagtaacagatgtatatgtcgacc

SEQ ID NO: 51 - DNA, 47 bp, synthetic construct
  source 1..47: mol_type = other DNA
  modified_base 6: mod_base = OTHER; note = 5-tryptamino-uracil
  modified_base 7: mod_base = OTHER; note = 5-tryptamino-uracil
  modified_base 18: mod_base = OTHER; note = 5-tryptamino-uracil
  modified_base 19: mod_base = OTHER; note = 5-tryptamino-uracil
  modified_base 22: mod_base = OTHER; note = 5-tryptamino-uracil
  modified_base 24: mod_base = OTHER; note = 5-tryptamino-uracil
  modified_base 25: mod_base = OTHER; note = 5-tryptamino-uracil
  modified_base 29: mod_base = OTHER; note = 5-tryptamino-uracil
  modified_base 30: mod_base = OTHER; note = 5-tryptamino-uracil
  modified_base 33: mod_base = OTHER; note = 5-tryptamino-uracil
  modified_base 34: mod_base = OTHER; note = 5-tryptamino-uracil
  modified_base 43: mod_base = OTHER; note = 5-tryptamino-uracil
  modified_base 45: mod_base = OTHER; note = 5-tryptamino-uracil
ccccattcagacaacccttggtgttcaattcattgcaacgagtgtcc

SEQ ID NO: 52 - DNA, 47 bp, synthetic construct
  source 1..47: mol_type = other DNA
  modified_base 4: mod_base = OTHER; note = 5-tryptamino-uracil
  modified_base 9: mod_base = OTHER; note = 5-tryptamino-uracil
  modified_base 11: mod_base = OTHER; note = 5-tryptamino-uracil
  modified_base 13: mod_base = OTHER; note = 5-tryptamino-uracil
  modified_base 14: mod_base = OTHER; note = 5-tryptamino-uracil
  modified_base 19: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 34: mod_base = OTHER; note = 5-tryptamino-uracil
  modified_base 37: mod_base = OTHER; note = 5-tryptamino-uracil
  modified_base 42: mod_base = OTHER; note = 5-tryptamino-uracil
  modified_base 43: mod_base = OTHER; note = 5-tryptamino-uracil
ccatcacatctattgaggtttacccttgacgcgtcataaacttcacc

SEQ ID NO: 53 - DNA, 47 bp, synthetic construct
  source 1..47: mol_type = other DNA
  modified_base 6: mod_base = OTHER; note = 5-tryptamino-uracil
  modified_base 10: mod_base = OTHER; note = 5-tryptamino-uracil
  modified_base 12: mod_base = OTHER; note = 5-tryptamino-uracil
  modified_base 13: mod_base = OTHER; note = 5-tryptamino-uracil
  modified_base 20: mod_base = OTHER; note = 5-tryptamino-uracil
  modified_base 25: mod_base = OTHER; note = 5-tryptamino-uracil
  modified_base 28: mod_base = OTHER; note = 5-tryptamino-uracil
  modified_base 31: mod_base = OTHER; note = 5-tryptamino-uracil
  modified_base 39: mod_base = OTHER; note = 5-tryptamino-uracil
  modified_base 41: mod_base = OTHER; note = 5-tryptamino-uracil
  modified_base 44: mod_base = OTHER; note = 5-tryptamino-uracil
ccccctcagtgttaggaggtagaatactcctcaacacatctcgtccc

SEQ ID NO: 54 - DNA, 47 bp, synthetic construct
  source 1..47: mol_type = other DNA
  modified_base 3: mod_base = OTHER; note = 5-tryptamino-uracil
  modified_base 4: mod_base = OTHER; note = 5-tryptamino-uracil
  modified_base 8: mod_base = OTHER; note = 5-tryptamino-uracil
  modified_base 11: mod_base = OTHER; note = 5-tryptamino-uracil
  modified_base 14: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 21: mod_base = OTHER; note = 5-tryptamino-uracil
  modified_base 26: mod_base = OTHER; note = 5-tryptamino-uracil
  modified_base 27: mod_base = OTHER; note = 5-tryptamino-uracil
  modified_base 29: mod_base = OTHER; note = 5-tryptamino-uracil
  modified_base 32: mod_base = OTHER; note = 5-tryptamino-uracil
  modified_base 35: mod_base = OTHER; note = 5-tryptamino-uracil
  modified_base 38: mod_base = OTHER; note = 5-tryptamino-uracil
  modified_base 40: mod_base = OTHER; note = 5-tryptamino-uracil
  modified_base 42: mod_base = OTHER; note = 5-tryptamino-uracil
  modified_base 43: mod_base = OTHER; note = 5-tryptamino-uracil
ccttcagtcctcatattaggtagacttctggtcctaatatattcacc

SEQ ID NO: 55 - DNA, 47 bp, synthetic construct
  source 1..47: mol_type = other DNA
  modified_base 5: mod_base = OTHER; note = 5-tryptamino-uracil
  modified_base 6: mod_base = OTHER; note = 5-tryptamino-uracil
  modified_base 8: mod_base = OTHER; note = 5-tryptamino-uracil
  modified_base 9: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 17: mod_base = OTHER; note = 5-tryptamino-uracil
  modified_base 18: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 26: mod_base = OTHER; note = 5-tryptamino-uracil
  modified_base 32: mod_base = OTHER; note = 5-tryptamino-uracil
  modified_base 35: mod_base = OTHER; note = 5-tryptamino-uracil
  modified_base 39: mod_base = OTHER; note = 5-tryptamino-uracil
  modified_base 42: mod_base = OTHER; note = 5-tryptamino-uracil
  modified_base 44: mod_base = OTHER; note = 5-tryptamino-uracil
  modified_base 45: mod_base = OTHER; note = 5-tryptamino-uracil
cccattgttagaattgttaaattcctgacagtcgtcgataatgttcc

SEQ ID NO: 56 - DNA, 47 bp, synthetic construct
  source 1..47: mol_type = other DNA
  modified_base 3: mod_base = OTHER; note = 5-tryptamino-uracil
  modified_base 5: mod_base = OTHER; note = 5-tryptamino-uracil
  modified_base 7: mod_base = OTHER; note = 5-tryptamino-uracil
  modified_base 9: mod_base = OTHER; note = 5-tryptamino-uracil
  modified_base 13: mod_base = OTHER; note = 5-tryptamino-uracil
  modified_base 16: mod_base = OTHER; note = 5-tryptamino-uracil
  modified_base 17: mod_base = OTHER; note = 5-tryptamino-uracil
  modified_base 20: mod_base = OTHER; note = 5-tryptamino-uracil
  modified_base 24: mod_base = OTHER; note = 5-tryptamino-uracil
  modified_base 26: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 36: mod_base = OTHER; note = 5-tryptamino-uracil
  modified_base 37: mod_base = OTHER; note = 5-tryptamino-uracil
  modified_base 39: mod_base = OTHER; note = 5-tryptamino-uracil
  modified_base 40: mod_base = OTHER; note = 5-tryptamino-uracil
cctgtgtatggctcgttgctggatctgataggtgcttcttccacccc

SEQ ID NO: 57 - DNA, 47 bp, synthetic construct
  source 1..47: mol_type = other DNA
  modified_base 5: mod_base = OTHER; note = 5-tryptamino-uracil
  modified_base 8: mod_base = OTHER; note = 5-tryptamino-uracil
  modified_base 11: mod_base = OTHER; note = 5-tryptamino-uracil
  modified_base 12: mod_base = OTHER; note = 5-tryptamino-uracil
  modified_base 13: mod_base = OTHER; note = 5-tryptamino-uracil
  modified_base 16: mod_base = OTHER; note = 5-tryptamino-uracil
  modified_base 20: mod_base = OTHER; note = 5-tryptamino-uracil
  modified_base 21: mod_base = OTHER; note = 5-tryptamino-uracil
  modified_base 23: mod_base = OTHER; note = 5-tryptamino-uracil
  modified_base 25: mod_base = OTHER; note = 5-tryptamino-uracil
  modified_base 36: mod_base = OTHER; note = 5-tryptamino-uracil
  modified_base 38: mod_base = OTHER; note = 5-tryptamino-uracil
  modified_base 45: mod_base = OTHER; note = 5-tryptamino-uracil
ccaatgatactttactggattatgtaaaacgcgcctatacaaagtcc

SEQ ID NO: 58 - DNA, 47 bp, synthetic construct
  source 1..47: mol_type = other DNA
  modified_base 5: mod_base = OTHER; note = 5-tryptamino-uracil
  modified_base 7: mod_base = OTHER; note = 5-tryptamino-uracil
  modified_base 12: mod_base = OTHER; note = 5-tryptamino-uracil
  modified_base 15: mod_base = OTHER; note = 5-tryptamino-uracil
  modified_base 19: mod_base = OTHER; note = 5-tryptamino-uracil
  modified_base 21: mod_base = OTHER; note = 5-tryptamino-uracil
  modified_base 31: mod_base = OTHER; note = 5-tryptamino-uracil
  modified_base 34: mod_base = OTHER; note = 5-tryptamino-uracil
  modified_base 35: mod_base = OTHER; note = 5-tryptamino-uracil
  modified_base 37: mod_base = OTHER; note = 5-tryptamino-uracil
  modified_base 39: mod_base = OTHER; note = 5-tryptamino-uracil
  modified_base 41: mod_base = OTHER; note = 5-tryptamino-uracil
  modified_base 42: mod_base = OTHER; note = 5-tryptamino-uracil
  modified_base 43: mod_base = OTHER; note = 5-tryptamino-uracil
ccaatatgcgatgatgaatgtcagagcagctcattgtgtatttgacc

SEQ ID NO: 59 - DNA, 47 bp, synthetic construct
  source 1..47: mol_type = other DNA
  modified_base 3: mod_base = OTHER; note = 5-tryptamino-uracil
  modified_base 7: mod_base = OTHER; note = 5-tryptamino-uracil
  modified_base 8: mod_base = OTHER; note = 5-tryptamino-uracil
  modified_base 12: mod_base = OTHER; note = 5-tryptamino-uracil
  modified_base 13: mod_base = OTHER; note = 5-tryptamino-uracil
  modified_base 16: mod_base = OTHER; note = 5-tryptamino-uracil
  modified_base 18: mod_base = OTHER; note = 5-tryptamino-uracil
  modified_base 20: mod_base = OTHER; note = 5-tryptamino-uracil
  modified_base 25: mod_base = OTHER; note = 5-tryptamino-uracil
  modified_base 26: mod_base = OTHER; note = 5-tryptamino-uracil
  modified_base 29: mod_base = OTHER; note = 5-tryptamino-uracil
  modified_base 33: mod_base = OTHER; note = 5-tryptamino-uracil
  modified_base 35: mod_base = OTHER; note = 5-tryptamino-uracil
  modified_base 38: mod_base = OTHER; note = 5-tryptamino-uracil
  modified_base 39: mod_base = OTHER; note = 5-tryptamino-uracil
  modified_base 40: mod_base = OTHER; note = 5-tryptamino-uracil
  modified_base 45: mod_base = OTHER; note = 5-tryptamino-uracil
cctaccttaacttcgtatgtaaacttgatggatgtcatttacactcc

SEQ ID NO: 60 - DNA, 47 bp, synthetic construct
  source 1..47: mol_type = other DNA
  modified_base 3: mod_base = OTHER; note = 5-tryptamino-uracil
  modified_base 6: mod_base = OTHER; note = 5-tryptamino-uracil
  modified_base 7: mod_base = OTHER; note = 5-tryptamino-uracil
  modified_base 9: mod_base = OTHER; note = 5-tryptamino-uracil
  modified_base 10: mod_base = OTHER; note = 5-tryptamino-uracil
  modified_base 14: mod_base = OTHER; note = 5-tryptamino-uracil
  modified_base 15: mod_base = OTHER; note = 5-tryptamino-uracil
  modified_base 22: mod_base = OTHER; note = 5-tryptamino-uracil
  modified_base 23: mod_base = OTHER; note = 5-tryptamino-uracil
  modified_base 29: mod_base = OTHER; note = 5-tryptamino-uracil
  modified_base 31: mod_base = OTHER; note = 5-tryptamino-uracil
  modified_base 36: mod_base = OTHER; note = 5-tryptamino-uracil
  modified_base 37: mod_base = OTHER; note = 5-tryptamino-uracil
  modified_base 40: mod_base = OTHER; note = 5-tryptamino-uracil
  modified_base 41: mod_base = OTHER; note = 5-tryptamino-uracil
  modified_base 44: mod_base = OTHER; note = 5-tryptamino-uracil
cctgcttgttaacttgaaaagttacaactgtaaacttgcttactgcc

(19) United States
(12) Patent Application Publication
(10) Pub. No.: US 2026/0250660 A1
(43) Pub. Date: Aug. 27, 2026

(54) BIO-FLUID'S LIGAND LIBRARY ENRICHMENT TECHNOLOGY AND USES THEREOF

(71) Applicant: Eisai R&D Management Co., Ltd., Tokyo (JP)

(72) Inventor: Yogo Sakakibara, Kobe-Shi, Hyogo (JP)

(73) Assignee: Eisai R&D Management Co., Ltd., Tokyo (JP)

(21) Appl. No.: 18/872,785

(22) PCT Filed: Jul. 3, 2023
(86) PCT No.: PCT/JP2023/024651

§ 371 Date: Dec. 6, 2024

Related U.S. Application Data
(60) Provisional application No. 63/358,354, filed on Jul. 5, 2022.

(51) Int. Cl. C12N 15/10; G16B 35/20

(52) U.S. Cl. CPC C12N 15/1048; G16B 35/20

(57) ABSTRACT

The present disclosure, at least in part, provides methods for generating aptamer libraries capable from binding to multiple target molecules (e.g., one or more biomolecules) in a complexed sample (e.g., biological sample) using electrophoresis mobility shift (e.g., 2-dimensional electrophoresis). The present disclosure also provides databases comprising information from one or more aptamer library (e.g., aptamer library generated by the methods described herein) and methods of utilizing such aptamer libraries (e.g., by comparing the information of a reference aptamer library and a target aptamer library to discern the differences).

### TECHNICAL FIELD

The present disclosure generally relates to a method for enriching a plurality of aptamers capable of binding to one or more biomolecules in a biological sample.

### BACKGROUND ART

Systematic Evolution of Ligands by EXponential Enrichment (SELEX) is a common method currently used for isolating high-affinity aptamers to a target molecule from a large pool with random sequences. Aptamers can be selected against a broad range of targets. Aptamers have high affinity and specificity to their target molecules. However, many SELEX selection methods have been developed for isolating aptamers targeting a single molecule. Due to the low successful rates for aptamer selection, many factors in selection process remains elusive.

### CITATION LIST

### Patent Literature

[PTL 1] US 2006/0160763

### Non Patent Literature

[NPL 1] Keefe et al., Aptamers as therapeutics. Nat. Rev. Drug Discov. 2010; 9:537-550.
[NPL 2] Jayasena S. D. Aptamers: An emerging class of molecules that rival antibodies in diagnostics. Clin. Chem. 1999; 45:1628-1650
[NPL 3] Tuerk et al., Systematic evolution of ligands by exponential enrichment: RNA ligands to bacteriophage T4 DNA polymerase. Science. 1990; 249:505-510.
NPL 4: Ellington et al., In vitro selection of RNA molecules that bind specific ligands. Nature. 1990; 346:818-822.
NPL 5: SELEX—A (Registered Trademark) evolutionary method to generate high-affinity nucleic acid ligands. Biomolecular Engineering, 24 (4): 381-403.
NPL 6: Klug & Famulok (1994). All you wanted to know about SELEX. Molecular Biology Reports, 20:97-107.
NPL 7: Darmostuk et al. (2015). Current approaches in SELEX: An update to aptamer selection technology. Biotechnology Advances, 33 (6): 1141-1161.
NPL 8: Morris et al., High affinity ligands from in vitro selection: Complex targets, Proc Natl Acad Sci USA. 1998 Mar. 17; 95 (6): 2902-2907.
NPL 9: Duffy et al., Modified nucleic acids: replication, evolution, and next-generation therapeutics, BMC Biology volume 18, Article number: 112 (2020).
NPL 10: Hollenstein (2015) Generation of long, fully modified, and serum-resistant oligonucleotides by rolling circle amplification. Organic Biomolecular Chemistry, 13:9829.
NPL 11: Kong et al. (2016). Generation of Synthetic Copolymer Libraries by Combinatorial Assembly on Nucleic Acid Templates. ACS Combinatorial Science, 18:355-370.
NPL 12: Abraham et al., A quick and effective in-house method of DNA purification from agarose gel, suitable for sequencing, 3 Biotech. 2017 July; 7 (3): 180.
NPL 13: Leonard et al., Basic Methods in Molecular Biology, 1986, SECTION 5-5-Agarose Gel Electrophoresis, pages 58-61.
NPL 14: Chai et al., Principle of Emulsion PCR and Its Applications in Biotechnology, J Anim Reprod Biotechnol 2019; 34:259-266.
NPL 15: Shao et al., Emulsion PCR: A High Efficient Way of PCR Amplification of Random DNA Libraries in Aptamer Selection, PLOS ONE 6 (9): e24910.

### SUMMARY OF INVENTION

### Technical Problem

The object of the present disclosure is to provide methods for enriching a plurality of aptamers capable of binding to one or more biomolecules in a biological sample. Another objective of the present disclosure is to provide methods for profiling a target aptamer library for a target biological sample.

### Solution to Problem

The present disclosure, at least in part, provides methods for generating aptamer libraries comprising a plurality of aptamers capable from binding to multiple target molecules (e.g., one or more biomolecules) in a complex sample (e.g., biological sample) using electrophoresis mobility shift (e.g., 2-dimensional electrophoresis). In some embodiments, the methods described herein are described as BIO-fluid's Ligand Library Enrichment Technology (BIOLLET). The present disclosure also provides databases comprising information from one or more aptamer libraries (e.g., aptamer library generated by the methods described herein) and methods of utilizing such aptamer libraries (e.g., by comparing the information of a reference aptamer library and a target aptamer library to discern the differences).

In some aspects, the present disclosure provides a method for enriching a plurality of aptamers capable of binding to one or more biomolecules in a biological sample, the method comprising: (i) contacting a plurality of candidate aptamers with the biological sample to form a composition comprising a plurality of aptamer-biomolecule complexes; (ii) subjecting the composition to electrophoresis in a first electrophoresis medium in a first direction to obtain a portion of the first electrophoresis medium that comprises the plurality of aptamer-biomolecule complexes; (iii) subjecting the portion of the first electrophoresis medium to electrophoresis in a second electrophoresis medium in a second direction to obtain a portion of the second electrophoresis medium that comprises the plurality of aptamer-biomolecule complexes; and (iv) extracting the plurality of aptamers capable of binding to biomolecules in the biological sample from the plurality of aptamer-biomolecule complexes.

In some embodiments, the method further comprising (v) amplifying the plurality of aptamers capable of binding to biomolecules in the biological samples to form an aptamer library.

In some embodiments, wherein the plurality of aptamers capable of binding to biomolecules in the biological samples are amplified by polymerase chain reaction (PCR). In some embodiments, the PCR is emulsion PCR.

In some embodiments, the first electrophoresis medium is a first agarose gel. In some embodiments, the second electrophoresis medium is a second agarose gel.

In some embodiments, the first and the second electrophoresis media comprise sodium ion, potassium ion, lithium ion, ammonium ion or any combination thereof at a concentration of between 100 mM and 200 mM. In some embodiments, the sodium ion is in the form of sodium chloride. In some embodiments, the first and the second electrophoresis media comprise magnesium ion, calcium ion, cupper ion, zinc ion or any combination thereof at a concentration of 10 mM or less. In some embodiments, the first and the second electrophoresis media comprise magnesium ion, calcium ion, cupper ion, zinc ion or any combination thereof at a concentration of between 0.5 mM and 2 mM. In some embodiments, the first and the second electrophoresis media comprise magnesium ion, calcium ion, cupper ion, zinc ion or any combination thereof at a concentration of 1 mM. In some embodiments, the magnesium ion is in the form of magnesium chloride.

In some embodiments, step (ii) further comprises excising the portion of the first electrophoresis medium comprising the plurality of aptamer-biomolecule complexes from the rest of the first electrophoresis medium. In some embodiments, the portion of the first electrophoresis medium is fitted to a well in the second electrophoresis medium for performing the electrophoresis in the second direction.

In some embodiments, the electrophoresis in the first direct and the second direction is performed in a temperature between 10° C. and 20° C.

In some embodiments, the biological sample is serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, or cell lysates. In some embodiments, the biomolecules in the biological sample comprise nucleic acids, proteins, polypeptides, carbohydrates, lipids, or a combination thereof. In some embodiments, the biological sample is not denatured. In some embodiments, the biological sample is diluted at a ratio between 1:200 and 1:2 prior to contacting with the plurality of candidate aptamers.

In some embodiments, the method further comprising contacting the biological sample to a plurality of competitor nucleic acids prior to contacting with the plurality of candidate aptamers. In some embodiments, the plurality of competitor nucleic acids are a random set of unrelated nucleic acids. In some embodiments, the plurality competitor nucleic acids are salmon sperm DNA.

In some embodiments, wherein the plurality of candidate aptamers are single stranded DNAs (ssDNA), double stranded DNAs (dsDNA), single stranded RNAs, or peptides. In some embodiments, the plurality of candidate aptamers are single stranded DNAs (ssDNA).

In some embodiments, each of the plurality of the candidate aptamers comprises modified nucleotide. In some embodiments, the each of the plurality of the candidate aptamers comprises one or more 5-tryptamino-uracil in place of thymine.

In some embodiments, each of the plurality of the candidate aptamers comprises a detectable label. In some embodiments, the detectable label is a fluorescent molecule.

In some embodiments, the method further comprising excising the portion of the second electrophoresis medium containing the plurality of aptamer-biomolecule complexes from the rest of the second electrophoresis medium and extracting the plurality of aptamer-biomolecule complexes from the portion of the second electrophoresis medium prior to step (iv).

In some embodiments, the steps (i)-(v) are repeated at least 4 times, and wherein the aptamer library obtained in step (v) is used as the starting plurality of candidate aptamers when repeating step (i). In some embodiments, the (i)-(iv) are repeated at least 9 times, and wherein the aptamer library obtained in step (iv) is used as the starting plurality of candidate aptamers when repeating step (i)

In some embodiments, the method further comprising identifying the sequence of the plurality of aptamers capable of binding to biomolecules in the biological sample. In some embodiments, identifying the sequence of the plurality of aptamers capable of binding to biomolecules in the biological sample comprises next generation sequencing (NGS).

In some embodiments, the method further comprising identifying the biomolecules bound to the plurality of aptamers capable of binding to biomolecules in the biological sample.

In some embodiments, the method further comprising compiling and storing the sequence information of the aptamer library capable of binding to biomolecules in the biological sample and/or the information of the biomolecules bound to aptamers in the aptamer library to a computer readable format.

In some aspects, the present disclosure further comprises an aptamer database comprising information of one or more aptamer libraries generated by the method described herein, wherein each aptamer library comprises the aptamers capable of binding to biomolecules in a biological sample.

In some aspects, the present disclosure provides a method for profiling a target aptamer library for a target biological sample, the method comprising: (i) acquiring the information of the target aptamer library for the target biological sample from a first aptamer database, (ii) acquiring the information of a reference aptamer library for a reference biological sample from a second aptamer database, and (iii) comparing the information of the target aptamer library for the target biological sample and the reference aptamer library for the reference biological sample thereby profiling the target aptamer library

In some aspects, the present disclosure provides a method for diagnosing diseases in a target subject, the method comprising: (i) acquiring the information of the target aptamer library for the target biological sample from a first aptamer database, (ii) acquiring the information of a reference aptamer library for a reference biological sample from a second aptamer database, and (iii) comparing the information of the target aptamer library for the target biological sample and the reference aptamer library for the reference biological sample.

In some aspects, the present disclosure provides a method for assisting the diagnosis of diseases in a target subject, the method comprising: (i) acquiring the information of the target aptamer library for the target biological sample from a first aptamer database, (ii) acquiring the information of a reference aptamer library for a reference biological sample from a second aptamer database, and (iii) comparing the information of the target aptamer library for the target biological sample and the reference aptamer library for the reference biological sample.

In some aspects, the present disclosure provides a method for determining the state of physical condition in a target subject, the method comprising: (i) acquiring the information of the target aptamer library for the target biological sample from a first aptamer database, (ii) acquiring the information of a reference aptamer library for a reference biological sample from a second aptamer database, and (iii) comparing the information of the target aptamer library for the target biological sample and the reference aptamer library for the reference biological sample.

In some aspects, the present disclosure provides a non-transitory computer-readable medium having stored thereon instructions that, when executed by at least one processor, cause the at least one processor to perform a method for profiling a target aptamer library for a target biological sample, the method comprising: (i) acquiring the information of the target aptamer library for the target biological sample from a first aptamer database, (ii) acquiring the information of a reference aptamer library for a reference biological sample from a second aptamer database, and (iii) comparing the information of the target aptamer library for the target biological sample and the reference aptamer library for the reference biological sample thereby profiling the target aptamer library.

In some embodiments, the first aptamer database in step (i) is an aptamer database described herein. In some embodiments, the second aptamer database in step (ii) is an aptamer database described herein. In some embodiments, the first aptamer database in step (i) is an aptamer database and the second aptamer database in step (ii) is an aptamer database described herein. In some embodiments, the first aptamer database and the second aptamer database are the same. In some embodiments, the first aptamer database and the second aptamer database are different.

In some embodiments, step (i) comprises downloading the information of the target aptamer library for the target biological sample from the aptamer database to a user computer or extracting the information of the target aptamer library for the target biological sample from the aptamer database to a server where the aptamer database is stored.

In some embodiments, step (ii) comprises downloading the information of the reference aptamer library for the reference biological sample from the aptamer database to a user computer or extracting the information of the reference aptamer library for the reference biological sample from the aptamer database to a server where the aptamer database is stored.

In some embodiments, the method further comprising step (iv) determining the difference between the information of the target aptamer library and the reference aptamer library. In some embodiments, step (iv) comprises identifying biomolecules bound to aptamers in the target aptamer library or the reference aptamer library that show the difference between the information of the target aptamer library and the reference aptamer library.

In some embodiments, the target biological sample is a biological sample from a subject of the same genetic background as the reference biological sample. In some embodiments, the target biological sample is a biological sample from a subject of a different genetic background as the reference biological sample. In some embodiments, the target biological sample is from a target subject having or suspected of having a disease. In some embodiments, the reference biological sample is from a healthy subject. In some embodiments, the comparing step results in determining whether the target subject has the disease.

In some embodiments, the target biological sample is a diseased sample. In some embodiments, the reference biological sample is a non-diseased sample. In some embodiments, the reference biological sample is a diseased sample. In some embodiments, comparing step results in identifying biomarkers for the diseased sample.

In some embodiments, the biological sample is serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, or cell lysates.

### Advantageous Effects of Invention

The present disclosure provides a method for enriching a plurality of aptamers capable of binding to one or more biomolecules in a biological sample. The method enables selecting aptamers capable of binding to one or more biomolecules at high successful rates. Furthermore, the present disclosure provides a method for profiling a target aptamer library. The method enables determining the state of physical condition in a target subject or diagnosing diseases in a target subject.

### BRIEF DESCRIPTION OF DRAWINGS

FIG. 1 Gel imaging data after 1D- and 2D-electrophoresis after round 1 selection are shown. In the gel imaging data of 1D-electrophoresis, the left lane shows control data running without sample and the right lane shows the gel shift data.

FIG. 2 Gel imaging data after 1D-electrophoresis at round 5 is shown. The left lane shows control gel shift data without competitors and the center lane shows the gel shift data. The arrows at the right of the gel image shows the gel excision points to separate top and bottom gels to check different sequence evolutions in different gel shift regions.

FIG. 3 Growth curves of aptamer library enrichment after various rounds of selection are shown. Normalized result size indicates the number of different aptamer sequences observed in next generation sequencing analysis with frequency more than 3 reads.

FIG. 4 Result of comparative analysis of information of aptamer libraries for serum samples from different C57BL/6N mice. Top30 sequences in the result of C57BL/6N serum B was selected and shown in the figure. Sequence #10 and #15 showed C57BL/6N serum D specificity.

FIG. 5 Result of comparative analysis of information of aptamer libraries for C57BL/6N and BALB/c serum samples. Top30 sequences in the result of C57BL/6N serum B was selected and shown in the figure. Sequence #14 and #27 showed no reactivity toward BALB/c serum.

FIG. 6 SDS-PAGE data for aptamer-based precipitation assay by using sd-10 aptamer. The sequence of sd-10 aptamer is shown in the table. Small letter “t” indicates 5-tryptamino-uracil. Arrowhead shows a target protein band for sd10 aptamer.

FIG. 7 SDS-PAGE data for aptamer-based precipitation assay by using Nb-01 aptamer. The sequence of Nb-01 aptamer is shown in the table. Small letter “t” indicates 5-tryptamino-uracil. Arrowhead shows a target protein band for Nb-01 aptamer

FIG. 8 Western-blotting analysis of sd-10 aptamer target protein by using anti-mouse IgG antibody.

FIG. 9 Western-blotting analysis of Nb-01 aptamer target protein by using antiGPLD-1 antibody.

FIG. 10 Comparative analysis of Information of Aptamer Libraries for human CSF samples derived from HC and AD patients.

### DESCRIPTION OF EMBODIMENTS

The present disclosure, at least in part, provides methods for generating aptamer libraries comprising a plurality of aptamers capable of from binding to multiple target molecules (e.g., one or more biomolecules) in a complex sample (e.g., biological sample) using two-dimensional electrophoresis mobility shift (e.g., 2-D electrophoresis). The present disclosure also provides databases comprising information from one or more aptamer library (e.g., aptamer library generated by the methods described herein) and methods of utilizing such aptamer libraries (e.g., by comparing the information of a reference aptamer library and a target aptamer library to discern the differences).

In some aspects, the present disclosure relates to methods for enriching a plurality of aptamers which are capable of binding to one or more target molecules (e.g. biomolecules) present in a sample (e.g., a biological sample).

The term “aptamer” as used herein, refers to oligonucleotides (e.g., single-stranded DNA (ssDNA) or single-stranded RNA (ssRNA) molecules that can selectively bind to a target molecule. In some embodiments, an aptamer is a nucleic acid aptamer. In some embodiments, an aptamer is a single-stranded DNA aptamer. In some embodiments, a nucleic acid aptamer comprises between 20 and 60 nucleotides, between 25 and 55 nucleotides, between 30 and 50 nucleotides, between 35 and 45 nucleotides, between 20 and 50 nucleotides, between 20 and 40 nucleotides, between 25 and 40 nucleotides, between 20 and 30 nucleotides, between 30 and 40 nucleotides, between 30 and 60 nucleotides, between 40 and 60 nucleotides, or between 50 and 60 nucleotides. In some embodiments, a target molecule of an aptamer includes proteins, peptides, carbohydrates, small molecules, toxins, and cells (e.g., live cells). An aptamers binds to its target with high affinity, selectivity and specificity (see., e.g., Non Patent Literature 1 and 2). Rather than primary sequence, aptamer binding is determined by its tertiary structure. Target recognition and binding involve three-dimensional, shape-dependent interactions as well as hydrophobic interactions, base-stacking, and intercalation. Aptamers offer advantages over antibodies as they can be engineered completely in vitro, are readily produced by chemical synthesis, possess desirable storage properties, and elicit little or no immunogenicity in therapeutic applications.

The concept of in vitro evolution of aptamers was introduced in 1990 and termed Systematic Evolution of Ligands by EXponential enrichment, or SELEX (Non Patent Literature 3-7). SELEX combines the rules of combinatorial library screening with in vitro evolution for enriching aptamers (e.g., DNA aptamers) against a wide range of target molecules. The SELEX process involves three interconnected steps: (i) the repeated incubation of a plurality of candidate aptamers with a target molecule to allow binding of high-affinity aptamers; (ii) the separation of high- from low-affinity binders and/or non-binders; and (iii) amplification of high-affinity binders utilizing polymerase chain reaction (PCR). This process is repeated until the high-affinity aptamers are enriched in the selection pool. While SELEX is successful in generating aptamers towards a purified protein, there is a possibility that these aptamers cannot recognize the same protein at its endogenous levels or conditions in a cell. A variant of SELEX, termed “complex target SELEX”, has been developed to select aptamers targeting proteins in its native conformation on a red blood cell membrane. The protein and their associated aptamer were separated by one dimensional SDS electrophoresis (Non Patent Literature 8).

### I. BIO-Fluid's Ligand Library Enrichment Technology (BIOLLET)

In some aspects, the present disclosure provides a method for enriching a plurality of aptamers capable of binding to one or more biomolecules in a biological sample, the method comprising: (i) contacting a plurality of candidate aptamers with the biological sample to form a composition comprising a plurality of aptamer-biomolecule complexes; (ii) subjecting the composition to electrophoresis in a first electrophoresis medium in a first direction to obtain a portion of the first electrophoresis medium that comprises the aptamer-biomolecule complexes; (iii) subjecting the portion of the first electrophoresis medium to electrophoresis in a second electrophoresis medium in a second direction to obtain a portion of the second electrophoresis medium that comprises the aptamer-biomolecule complexes; and (iv) extracting the plurality of aptamers capable of binding to biomolecules in the biological sample from the plurality of aptamer-biomolecule complexes. The term “aptamer-biomolecule complex”, as used herein, refers to the molecular complex formed between an aptamer and its target molecule (e.g., biomolecule) via specific binding. As used herein, the term “specific binding” refers to the ability of a molecule (e.g., an aptamer) to bind to a binding partner (e.g., a target molecule) with a degree of affinity or avidity that enables the molecule to be used to distinguish the binding partner from an appropriate control in a binding assay or other binding context. With respect to an aptamer, the term, “specific binding”, refers to the ability of the aptamer to bind to a target molecule with a degree of affinity or avidity, compared with an appropriate reference target molecule or target molecules, that enables the aptamer to be used to distinguish the specific target molecule from others. In some embodiments, an aptamer specifically binds to a target molecule if the aptamer has a K_{D} for binding the target molecule of at least about 10⁻⁶ M, 10⁻⁷ M, 10−8 M, 10⁻⁹ M, 10⁻¹⁰ M, 10⁻¹¹ M, 10⁻¹² M, 10⁻¹³ M, or less.

The present disclosure recognizes the difficulties of enriching aptamers capable of binding to target molecules in their native conformation from a complex sample (e.g., a biological sample). The present disclosure also identifies the disadvantage of enriching aptamer-target molecule (e.g., biomolecule) complexes using 1-dimensional electrophoresis (1D-electrophoresis), and/or running electrophoresis under mild conditions (e.g., low salt conditions). For example, and not wishing to be bound to any particular theory, 1D-electrophoresis may not be able to separate aptamer-target molecule (e.g., biomolecule) complexes from aptamers bound to the target molecules due to non-specific binding. With respect to aptamer, as used herein, the term “non-specific binding”, refers to the ability of the aptamer to bind to a molecule with a degree of affinity or avidity, compared with an appropriate reference target molecule or target molecules, that does not enable the aptamer to be used to distinguish this molecule from others. In some embodiments, an aptamer non-specifically binds to a molecule if the aptamer has a K_{D} for binding the molecule of 10⁻⁶ M, 10⁻⁵ M, 10⁻⁴ M, 10⁻³ M, 10⁻² M, 10⁻¹ M, or higher.

In addition, the condition for 1D-electrophoresis may not be sufficient to break the non-specific binding between certain aptamers and target molecules. The present disclosure, therefore, sought to enriching a plurality of aptamers capable of binding to one or more biomolecules in a biological sample by enriching aptamer-target-molecule (e.g., biomolecule) complexes by 2D-electrophoresis. The 2D-electrophoresis can be performed under salt conditions sufficient to break the non-specific interaction between an aptamer and a target molecule but not sufficient to break the specific binding between aptamer and its target molecule. Accordingly, the present disclosure enables simultaneous enrichment of a plurality of aptamers capable of binding to multiple target molecules (e.g., biomolecules) in a complex sample (e.g., biological sample).

The term “candidate aptamers”, as used herein, refers to a pool of random aptamers or random oligonucleotides which can be aptamers to be subjected to the methods described herein for enrichment of aptamers capable of binding to one or more target molecules (e.g., biomolecules) from a sample (e.g., a biological sample). In some embodiments, in generating an initial plurality of candidate aptamers, the ratio of the nucleotides in each aptamer is optimized, for example, at an A:C:G:T molar ratio of 1.0:1.0:1.0:1.0, 1.5:1.5:1.0:1.2, 1.30:1.25:1.45:1.00, or 1.50:1.25:1.15:1.00. In some embodiments, the candidate aptamers are an initial pool of random aptamers or random oligonucleotides which can be aptamers that have not being selected. In some embodiments, the candidate aptamers are a resulting plurality of aptamers capable of binding to one or more target molecules (e.g., biomolecules) in a sample (e.g., biological sample) from the last round of enrichment using the method described herein. In some embodiments, the plurality of candidate aptamers are DNA or RNA. In some embodiments, the candidate aptamers are single-stranded DNA (ssDNA). In some embodiments, the plurality of the candidate aptamers are folded in their proper tertiary structure for binding the target molecules.

In some embodiments, the plurality of candidate aptamers comprises modified nucleotide. Modified nucleotide have been previously described, see, e.g., Non Patent Literature 9. In some embodiments, each of the plurality of candidate aptamers comprises at least one modified nucleotide. In some embodiments, each plurality of candidate aptamers comprises a modified nucleotide (e.g., modified nucleotide that can be used as a substrate by DNA polymerase). Modified nucleotides are well known in the art (see, e.g., Patent Literature 1, and Non Patent Literature 10 and 11). In some embodiments, each of the plurality of the candidate aptamers comprises, but are not limited to, for example, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, betaD-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 2,6-diaminopurine, or 5-tryptamino-uracil. In some embodiments, a modified nucleotide is a 2′-modified nucleotide. For example, the 2′-modified nucleotide may be a 2′-deoxy, 2′-fluoro, 2′-O-methoxyethyl, 2′-amino and 2′-aminoalkoxy modified nucleotides. In some embodiments, each of the plurality of the candidate aptamers comprises but not limited to one or more 5-tryptamino-uracil in place of thymine. In some embodiments, at least one thymine in each of the plurality of the candidate aptamers are replaced by a 5-tryptamino-uracil. In some embodiments, all of the thymine in each of the plurality of the candidate aptamers are replaced by a 5-tryptamino-uracil.

In some embodiments, each of the plurality of the candidate aptamers comprises a detectable label. The detectable label can facilitate detection of aptamer-target molecule (e.g., biomolecule) complexes. In some embodiments, the detectable label is a protein capable of generating a colorimetric change. In some embodiments, the protein capable of generating a colorimetric change is alkaline phosphatase, horseradish peroxidase, or luciferase. In some embodiments, the detectable label is a fluorescent molecule. In some embodiments, the fluorescent molecule includes but are not limited to fluorescent dye is TYE665, lucifer yellow, dansyl, TruRed, fluorescein, Cy2, Cy3, Cy7, TRITC, X-Rhodamine, or Texas red, green fluorescent protein (GFP), blue fluorescent protein (BFP), yellow fluorescent protein (YFP), red fluorescent protein (RFP), mCherry, mTurquoise2, or mOrange. In some embodiments, each of the plurality of the candidate aptamers is labeled at the 5′ end terminal phosphate group. In some embodiments, each of the plurality of the candidate aptamers is labeled at their 3′ end terminal hydroxyl group. In some embodiments, each of the plurality of the candidate aptamers comprises one or more than one detectable label. In some embodiments, each of the plurality of the candidate aptamers comprises a TYE665 fluorophore at the 5′ end.

In some embodiments, prior to contacting the target molecules (e.g., biomolecules) in a sample (e.g., biological sample), the plurality of the candidate aptamers are prepared in a selection buffer. In some embodiments, the selection buffer comprises nonidet-P40. In some embodiments, the selection buffer comprises nonidet-P40 at a concentration between 0.001% and 0.01%. In some embodiments, the selection buffer comprises nonidet-P40 at a concentration of 0.005%. In some embodiments, the selection buffer comprises a salt. In some embodiments, the salt is capable of stabilization of the tertiary structure of the aptamers. In some embodiments, the salt is a divalent ion including but not limited to magnesium ion, calcium ion, cupper ion, zinc ion or any combination thereof. In some embodiments, the selection buffer comprises magnesium ion at a concentration between 0.1 mM and 10 mM, between 1 mM and 10 mM, between 5 mM and 10 mM, between 8 mM and 10 mM, between 6 mM and 8 mM, between 2 mM and 8 mM, between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the selection buffer comprises magnesium at a concentration at 1 mM. In some embodiments, the magnesium ion is in the form of magnesium chloride. In some embodiments, the plurality of the candidate aptamers are prepared in the selection buffer at a concentration between 50 nM and 8000 nM, between 60 nM and 7000 nM, between 50 nM and 6000 nM, between 50 nM and 8000 nM, between 50 nM and 8000 nM, between 50 nM and 8000 nM, between 50 nM and 5000 nM, between 60 nM and 4000 nM, between 70 nM and 3000 nM, between 80 nM and 2000 nM, between 90 nM and 1000 nM, between 100 nM and 1000 nM, between 50 nM and 100 nM, between 100 nM and 200 nM, between 200 nM and 500 nM, between 500 nM and 1000 nM, between 1000 nM and 2000 nM, between 2000 nM and 3000 nM, between 3000 nM and 4000 nM, between 4000 nM and 5000 nM, between 1000 nM and 5000 nM, between 1000 nM and 2500 nM, or between 2500 nM and 5000 nM.

In some embodiments, enriching a plurality of aptamers is also described as selecting for a plurality of aptamers. The terms “enriching”, “enrichment”, or “enrichment process” is used interchangeably with selecting, selection, or selection process, respectively. In some embodiments, enriching a plurality of aptamers comprises selection for aptamers that are capable of binding to one or more target molecule (e.g., biomolecules) from those that do not bind to one or more target molecule(s) (e.g., biomolecules). In some embodiments, enriching a plurality of aptamers comprises selection for aptamers with higher affinity to one or more target molecule (e.g., biomolecules) relative to those with lower affinity to one or more target molecule(s) (e.g., biomolecules).

In some embodiments, the method described herein comprises contacting a plurality of candidate aptamers with a complex sample (e.g., biological sample) comprising a plurality of different target molecules. In some embodiments, a complex sample comprises a plurality of different target molecules of the same type (e.g., the complex sample contains a plurality of different proteins). In some embodiments, a complex sample contains a plurality of different types of target molecules (e.g., the complex sample contains proteins, nucleic acids, small molecules, toxins, etc). In some embodiments, a complex sample contains different types of target molecules and each type of target molecules further contains different individual target molecules (e.g., the complex sample contains a plurality of different proteins, a plurality of different nucleic acids, a plurality of different small molecules, and a plurality of different toxins, etc). Examples of a complex sample include but are not limited to biological sample (e.g., biological fluid such as serum), environmental sample (e.g., samples obtained from river, lake, pond, soil, atmosphere, outer space, etc.), manufacturing sample (e.g., samples obtained from of a bioreactor, samples obtained from a HPLC flow through fluid, samples contain intermediate of a small molecule drug, etc). In some embodiments, a complex sample is a biological sample. The term “biological sample”, as used herein, refers to samples obtained from a biological subject.

Examples of biological samples include but are not limited to, whole blood, interstitial fluid, skin, lymphatic fluid, bile, serum, plasma, cerebral-spinal fluid (CSF), urine, amniotic fluid, bone marrow, bronchoalveolar lavage fluid, buccal swab, feces, gastrointestinal fluid, liposuction sample, saliva, milk, nasal swab, peritoneal fluid, semen, sputum, synovial fluid, tears, vaginal fluid, tissue biopsy, autopsy samples, cells or cell lysates, cultured cell, tissue sample (e.g., tissue sample from a human, a non-human animal, plants, insects, fungi), or in vivo endothelial cells. In some embodiments, a biological sample comprises target molecules (e.g., biomolecules) such as nucleic acids (e.g., DNAs and RNAs), proteins, peptides, lipids, polysaccharides, proteoglycans, and glycolipids. In some embodiments, the biological sample is serum. In some embodiments, the biological sample is obtained from a human subject. In some embodiments, the biological sample is obtained from a non-human subject. Examples of non-human subjects include, but are not limited to, monkeys, mice, rats, rabbits, goats, sheep, dogs, birds, and fish. In some embodiments, the subject is a healthy subject. In some embodiments, the subject is a subject suffering, suspected of suffering from a disease, or at a risk of developing a disease.

In some embodiments, the target molecules (e.g., biomolecules) in a sample (e.g., biological sample) are denatured prior to contacting with a plurality of candidate aptamers. In some embodiments, the target molecules (e.g., biomolecules) in a sample (e.g., biological sample) are not denatured prior to contacting with a plurality of candidate aptamers. The term “denature”, “denaturation” or “denatured”, as used herein, refers to subjecting the sample (e.g., biological sample) to a condition that breaks linkages (e.g., disulfide bridges), bonds (e.g., hydrogen bonds, ionic bonds, etc.), and/or interactions (e.g., hydrophobic interactions) within one or more target molecules in the sample (e.g., biomolecules such as proteins) that are responsible for the highly ordered structure of the target molecule (e.g., biomolecules such as proteins, and/or nucleic acids) in its natural (native) state. In some embodiments, a denatured sample comprises a sample wherein the biomolecules (e.g., proteins or nucleic acids) within the sample have lost their quaternary, tertiary, and secondary structure thereby only retaining their primary structure (e.g., linear amino acid sequence or nucleic acid sequence) such that the biomolecules do not retain their respective structures and/or assigned functions. In some embodiments, a denatured sample comprises a sample wherein the biomolecules (e.g., proteins or nucleic acids) have been degraded to fragments such that the biomolecules no longer retain it their structures and/or assigned functions. As such, a denatured sample comprises biomolecules (e.g., proteins or nucleic acids) that do not retain their assigned function (e.g., binding capabilities, biological activity, etc.). Conversely, a non-denatured sample is one that contains target molecules (e.g., biomolecules) that retain their native conformations and their respective assigned functions. Methods for denaturing target molecules in a sample are known in the art, such as by heating, by treatment with alkali, acid, urea, or detergents, or by vigorous shaking. In some embodiments, the target molecules (e.g., biomolecules) are in their native conformation. In some embodiments, contacting a plurality of candidate aptamers with target molecules (e.g., biomolecules) in their native conformation is advantageous in that the selected aptamers are capable of binding to their target molecules (e.g., biomolecules) in other conditions (e.g., in vivo in a subject) than the conditions where the aptamers are selected. It will be understood by those of ordinary skill in the art that denaturation comprises subjecting the target molecules (e.g., biomolecules) in a sample (e.g., biological samples) to a condition (e.g., heating, sonicating, incubating in the presence of a detergent, etc.) sufficient for any change in native or natural structure of a target molecule (e.g. biomolecule such as a protein, DNA, RNA, toxin, or small molecule) that renders it incapable of performing its assigned function. The terms “not denatured”, as used herein, refers to maintaining the structures of the target molecules (e.g., biomolecules) in the sample (e.g., biological samples) sufficient to perform their assigned functions. In some embodiments, the candidate aptamers of the present disclosure can be contacted with a biological sample that is not denatured. In some embodiments, the candidate aptamers of the present disclosure can be contacted with a sample comprising biomolecules (e.g., protein, DNA, RNA, toxin, or small molecule) that retain their quaternary, tertiary, and secondary structures and their respective assigned functions.

In some embodiments, the sample (e.g., biological sample) is not diluted prior to contacting with a plurality of candidate aptamers. In some embodiments, the sample (e.g., biological sample) is diluted prior to contacting with a plurality of candidate aptamers. In some embodiments, the biological sample is diluted at a ratio of between 1:1000 and 1:1, between 1:900 and 1:2, between 1:800 and 1:2, between 1:700 and 1:2, between 1:600 and 1:2, between 1:500 and 1:2, between 1:400 and 1:2, between 1:300 and 1:2, between 1:200 and 1:2, between 1:100 and 1:2, between 1:50 and 1:2, between 1:25 and 1:2, between 1:10 and 1:2, between 1:5 and 1:2, between 1:500 and 1:50, between 1:500 and 1:100, between 1:500 and 1:200, between 1:200 and 1:100, between 1:200 and 1:50, between 1:200 and 1:10, between 1:100 and 1:50, between 1:100 and 1:10, between 1:100 and 1:5, or between 1:100 and 1:2 prior to contacting the biological sample with the plurality of candidate aptamers. In some embodiments, the biological sample is a serum, and the serum is diluted at a ratio of between 1:500 and 1:10, between 1:500 and 1:50, between 1:500 and 1:100, between 1:300 and 1:10, between 1:300 and 1:50, between 1:300 and 1:100, between 1:300 and 1:200, between 1:200 and 1:100, between 1:200 and 1:150, between 1:200 and 1:50, between 1:250 and 1:200, or between 1:200 and 1:100, between 1:200 and 1:150, or between 1:200. In some embodiments, the serum is diluted at a ratio of 1:200 prior to contacting the plurality of candidate aptamers. In some embodiments, the biological sample is a CSF, and the CSF is diluted at a ratio of between 1:50 and 1:2, between 1:40 and 1:2, between 1:30 and 1:2, between 1:20 and 1:2, between 1:10 and 1:2, between 1:5 and 1:2, between 1:4 and 1:2, between 1:3 and 1:2, between 1:20 and 1:5, or between 1:10 and 1:5. In some embodiments, the biological sample is diluted in any suitable dilution buffer prior to contacting it with the plurality of candidate aptamers. Non-limiting examples of the dilution buffer include Phosphate-buffered saline (PBS), Dulbecco's phosphate-buffered saline (DPBS), Hanks' Balanced Salt Solution (HBSS), Dulbecco's Modified Eagle Medium (DMEM). One of ordinary skill can select the suitable dilution buffer according to the biological sample being used.

In some embodiments, the method further comprising contacting the sample (e.g., biological sample) to a plurality of competitor nucleic acids prior to contacting the sample (e.g., biological sample) with a plurality of candidate aptamers. The competitor nucleic acids are used to block non-specific binding between aptamers and target molecules. In some embodiments, the competitor nucleic acids are a random set of unrelated nucleic acids. In some embodiments, the competitor nucleic acids are salmon sperm DNA.

In some embodiments, contacting a plurality of candidate aptamers with a sample (e.g., biological sample) results in binding of the aptamers to their target molecules (e.g., biomolecules) to produce a composition. In some embodiments, the composition comprises aptamer-target molecule (e.g., aptamer-biomolecule) complexes. In some embodiments, the composition comprises unbound aptamers. In some embodiments, the composition comprises unbound target molecules (e.g., unbound biomolecules). In some embodiments, the composition comprises aptamer-target molecule (e.g., aptamer-biomolecule) complexes, unbound aptamers, and/or unbound target molecules (e.g., unbound biomolecules). In some embodiments, the composition also comprises aptamers bound to biomolecules due to non-specific binding.

In some embodiments, the method described herein comprises separating the aptamer-target molecule (e.g., aptamer-biomolecule) complexes from the unbound aptamers. In some embodiments, the method described herein comprises separating the aptamer-target molecule (e.g., aptamer-biomolecule) complexes from the unbound aptamers. In some embodiments, the method described herein comprises separating the aptamer-target molecule (e.g., aptamer-biomolecule) complexes from aptamers bound to biomolecules due to non-specific binding. In some embodiments, the present methods comprise enriching the aptamer-target molecule (e.g., aptamer-biomolecule complexes) from the composition that also comprises unbound aptamer, and/or aptamers bound to the target-molecules due to non-specific binding.

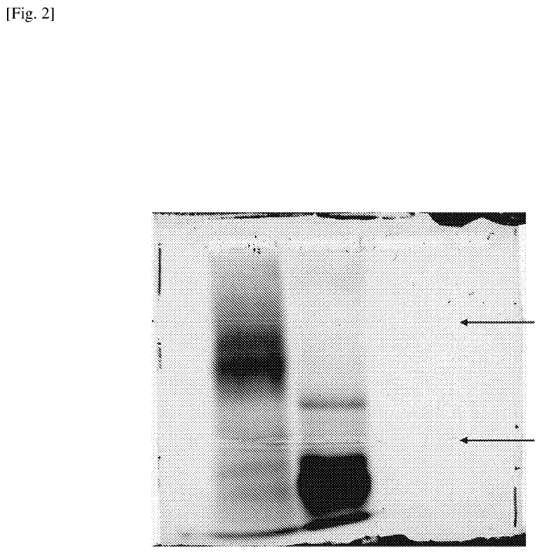

In some embodiments, enriching the aptamer-target molecule (e.g., aptamer-biomolecule complexes) from the composition that also comprises unbound aptamer, and/or aptamers bound to the target-molecules due to non-specific comprises subjecting the composition to electrophoresis. The term “electrophoresis”, as used herein, refers to a technique used to separate molecules (e.g., DNA, RNA, protein, aptamer-target molecule complexes) based on the size and electrical charge of the molecules being separated. An electrophoretic system includes two electrodes of opposite charge (anode, cathode), connected by a conducting electrophoresis medium. An electric current is used to move molecules to be separated through an electrophoresis medium. In some embodiments, a negatively charged is applied such that the molecules move towards a positive charge, Further, the electrophoresis medium usually contains pores that allow smaller molecules to move faster than larger molecules. In some embodiments, the present disclosure is based on the theory that the plurality of aptamer-target molecules (e.g., biomolecule) complexes are different in size from the unbound aptamer (e.g., biomolecules). In some embodiments, the unbound aptamers are smaller in size than the aptamer-target molecule (e.g., biomolecule) therefore migrate faster during electrophoresis. In some embodiments, the individual aptamer-target molecule (e.g., biomolecule) in the plurality of aptamer-target molecules (e.g., biomolecule) complexes are different in size from each other. In some embodiments, the individual aptamer-target molecule (e.g., biomolecule) in the plurality of aptamer-target molecule (e.g., biomolecule) complexes migrate at different speed during electrophoresis. In some embodiments, the methods described herein comprises subjecting the composition to electrophoresis in a first electrophoresis medium in a first direction. In some embodiments, the size difference between aptamer-target molecule (e.g., biomolecule) complexes and aptamers bound to the target molecules (e.g., biomolecules) due to non-specific binding is not sufficient to separate the aptamer-target molecule (e.g., biomolecule) complexes from the aptamers bound to the target molecules (e.g., biomolecules) due to non-specific binding. In some embodiments, after the electrophoresis in the first direction, a portion of the first electrophoresis medium comprises the aptamer-biomolecule complexes, and the same portion may also contain the aptamers bound to the target molecules (e.g., biomolecules) due to non-specific binding (FIG. 2C-2D).

Accordingly, in some embodiments, the present disclosure sought to enriching a plurality of aptamers capable of binding to one or more biomolecules in a biological sample by enriching aptamer-target molecule (e.g., biomolecule) complexes by 2D-electrophoresis. In some embodiments, the method comprising subjecting the portion of the first electrophoresis medium that comprises the aptamer-biomolecule complexes, which may also contain aptamers bound to the target molecules (e.g., biomolecules) due to non-specific binding to electrophoresis in a second electrophoresis medium in a second direction. The term “second direction” as used herein, refers to a direction different from the first direction. In some embodiments, a second direction is orthogonal to the first direction. In some embodiments, the method further comprising excising the portion of the first electrophoresis medium comprising the aptamer-biomolecule complexes (which may also contain aptamers bound to the target molecules (e.g., biomolecules) due to non-specific binding) from the rest of the first electrophoresis medium. In some embodiments, the method further comprises fit the excised portion of the first electrophoresis medium comprising the aptamer-biomolecule complexes (which may also contain aptamers bound to the target molecules (e.g., biomolecules) due to non-specific binding) in a well in the second electrophoresis medium. Subjecting the composition to electrophoresis in a second dimension breaks the weak, non-specific binding between aptamers and the target molecules (e.g., biomolecules), such that these aptamers become unbound aptamers and migrate faster than the aptamer-target molecule (e.g., biomolecule) complexes. In some embodiments, the aptamer-target molecule (e.g., biomolecule) complexes presents in the second electrophoresis medium in the diagonal region after electrophoresis in the second direction. In some embodiments, the method further comprising excising the portion of the second electrophoresis medium comprising the aptamer-biomolecule complexes from the rest of the second electrophoresis medium.

In some embodiments, the method further comprises extracting the aptamers capable of binding to one or more target molecules (e.g., biomolecules) from the portion of the second electrophoresis medium that contains the aptamer-biomolecule complexes. In some embodiments, the aptamers are separated from the aptamer-target molecule complexes during this step. Methods of extracting the aptamers from a portion of an electrophoresis medium has been previously described, e.g., by a commercially available kit such as Oligo Clean and Concentrator Kits by Zymo Research), QIAquick Gel Extraction Kit (QIAGEN, Hilden, Germany), Wizard SV Gel and PCR Clean Up System (Promega, Madison, WI, USA), and GENECLEAN (Registered Trademark) II kit (MP BIOMEDICALS, Solon, OH, USA), or by the method described in Non Patent Literature 12.

In some embodiments, an electrophoresis medium comprises pores allowing migration of the molecules being subjected to electrophoresis. Non-limiting examples of electrophoresis medium includes agarose, polyacrylamide, silica matrix, or starch. In some embodiments, the first electrophoresis medium is agarose. In some embodiments, the second electrophoresis medium is agarose. In some embodiments, the first electrophoresis medium is agarose, and the second electrophoresis medium is agarose. Methods of preparing an agarose gel for electrophoresis are known in the art, e.g., as described by Non Patent Literature 13. In some embodiments, the agarose gel is prepared from dry agarose power (e.g., by dissolving the agarose power in a suitable buffer such as Tris Buffer by heating, and letting the agarose solidify by cooling to room temperature). In some embodiments, the agarose gel is a pre-made gel purchased from a vendor. In some embodiments, the first electrophoresis medium is an agarose gel comprises agarose at a concentration of any concentration between 0.5% and 3% (e.g., 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5%), between 1%-2%, between 0.5%-1%, between 1.5%-2%, or between 2%-2.5%.

In some aspects, the present disclosure also contemplates the optimal conditions (e.g., salt, temperature) for performing the electrophoresis. In some embodiments, the 2D-electrophoresis is performed under a salt condition (e.g., the electrophoresis medium comprises an ion at a concentration) capable of mitigating electrostatic effect of biomolecules (e.g., proteins or DNAs). In some embodiments, the 2D-electrophoresis is performed under a salt condition (e.g., the electrophoresis medium comprises an ion at a concentration) sufficient to break the non-specific interaction between an aptamer and a target molecule but not sufficient to break the specific binding between aptamer and its target molecule. In some embodiments, such ion includes but are not limited to sodium ion, potassium ion, lithium ion, ammonium ion, or any combination thereof (e.g., combination of sodium ion and potassium ion; combination of sodium ion and lithium ion; combination of sodium ion and ammonium ion; combination of potassium ion and lithium ion; combination of potassium ion and ammonium ion; combination of lithium ion and ammonium ion; combination of sodium ion, potassium ion, and lithium ion; combination of sodium ion, potassium ion, and ammonium ion; combination of potassium ion, lithium ion, and ammonium ion; or combination of sodium ion, potassium ion, lithium ion, and ammonium ion). The term “any combination thereof at a concentration”, as used herein, refers to a total concentration of the ion(s) in a combination (e.g., any of the combination described herein). For example, a combination of two ions at a concentration between X and Y refers to a total concentration of the two ions is within the range of X and Y; a combination of three ions at a concentration of between X and Y refers to a total concentration of the three ions is within the range of X and Y, etc. A concentration of can be described using any unit known in the art, e.g., M, mM, UM, nM, pM, g/L, g/dL, g/mL, g/μL, g/nL, mg/L, mg/dL, mg/mL, mg/μL, mg/nL, μg/L, μg/dL, μg/mL, μg/μL, μg/nL, ng/L, ng/dL, ng/mL, ng/μL, ng/nL, pg/L, pg/dL, pg/mL, pg/μL, or pg/nL. For example, when an electrophoresis medium comprises a combination of ions (e.g., sodium, potassium, lithium, and/or ammonium ions) at a total concentration between 100 mM and 200 mM, it means that the total concentration of the ions thereof is between 100 mM and 200 mM. It is within the skill of one of ordinary skill in the art to select a concentration for each ion in the combination to reach a total concentration of a prescribed range.

In some embodiments, the first electrophoresis medium comprises sodium ion (e.g., sodium chloride), potassium ion, lithium ion, ammonium ion or any combination thereof at a concentration sufficient to break non-specific binding of aptamers to target-molecules. In some embodiments, the first electrophoresis medium comprises sodium ion (e.g., sodium chloride), potassium ion, lithium ion, ammonium ion or any combination thereof at a concentration of between 50 mM and 500 mM, between 80 mM and 450 mM, between 100 mM and 400 mM, between 150 mM and 350 mM, between 200 mM and 300 mM, between 100 mM and 400 mM, between 100 mM and 300 mM, between 100 mM and 200 mM, between 100 mM and 150 mM, between 150 mM and 200 mM, between 110 mM and 190 mM, between 120 mM and 180 mM, between 130 mM and 170 mM, between 140 mM and 160 mM, between 120 mM and 150 mM, between 120 mM and 160 mM, between 120 mM and 170 mM, between 120 mM and 130 mM, between 150 mM and 160 mM, between 150 mM and 170 mM, between 150 mM and 180 mM, between 150 mM and 190 mM, between 100 mM and 120 mM, between 120 mM and 130 mM, between 130 mM and 140 mM, between 140 mM and 150 mM, between 150 mM and 160 mM, between 160 mM and 170 mM, between 170 mM and 180 mM, between 180 mM and 190 mM, or between 190 mM and 200 mM. In some embodiments, the first electrophoresis medium comprises sodium ion (e.g., sodium chloride), potassium ion, lithium ion, ammonium ion or any combination thereof at a concentration of between 100 mM and 200 mM (e.g., any concentration between 100 mM and 200 mM).

In some embodiments, the second electrophoresis medium comprises sodium ion (e.g., sodium chloride), potassium ion, lithium ion, ammonium ion or any combination thereof at a concentration of between 50 mM and 500 mM, between 80 mM and 450 mM, between 100 mM and 400 mM, between 150 mM and 350 mM, between 200 mM and 300 mM, between 100 mM and 400 mM, between 100 mM and 300 mM, between 100 mM and 200 mM, between 100 mM and 150 mM, between 150 mM and 200 mM, between 110 mM and 190 mM, between 120 mM and 180 mM, between 130 mM and 170 mM, between 140 mM and 160 mM, between 120 mM and 150 mM, between 120 mM and 160 mM, between 120 mM and 170 mM, between 120 mM and 130 mM, between 150 mM and 160 mM, between 150 mM and 170 mM, between 150 mM and 180 mM, between 150 mM and 190 mM, between 100 mM and 120 mM, between 120 mM and 130 mM, between 130 mM and 140 mM, between 140 mM and 150 mM, between 150 mM and 160 mM, between 160 mM and 170 mM, between 170 mM and 180 mM, between 180 mM and 190 mM, or between 190 mM and 200 mM. In some embodiments, the second electrophoresis medium comprises sodium ion (e.g., sodium chloride), potassium ion, lithium ion, ammonium ion or any combination thereof at a concentration of between 100 mM and 200 mM (e.g., any concentration between 100 mM and 200 mM). In some embodiments, the ion is sodium ion. In some embodiments, the sodium ion can be any sodium salt known in the art (e.g., sodium chloride).

In addition, and in some embodiments, the 2D-electrophoresis is performed under salt conditions (e.g., a divalent ion at a concentration) sufficient to stabilize the structure of aptamers and/or the aptamer-target molecule (e.g., biomolecule) complexes. Non-limiting examples of divalent ion capable of stabilizing the structure of aptamers and/or the aptamer-target molecule (e.g., biomolecule) complexes include but are not limited to magnesium ion, calcium ion, copper ion, zinc ion, or any combination thereof. In some embodiments, the first electrophoresis medium comprises one or more divalent ion (e.g., magnesium ion, calcium ion, copper ion, zinc ion or any combination thereof) at a concentration sufficient to stabilize the structure of aptamers and/or the aptamer-target molecule (e.g., biomolecule) complexes. In some embodiments, the first electrophoresis medium comprises magnesium ion, calcium ion, copper ion, zinc ion or any combination thereof at a concentration of 10 mM or less (e.g., less than 10 mM, less than 9 mM, less than 8 mM, less than 7 mM, less than 6 mM, less than 5 mM, less than 4 mM, less than 3 mM, less than 2 mM, less than 1 mM, or less than 0.5 mM). In some embodiments, the first electrophoresis medium comprises magnesium ion, calcium ion, copper ion, zinc ion or any combination thereof at a concentration of between 0.1 mM and 10 mM, between 0.1 mM and 9 mM, between 0.1 mM and 8 mM, between 0.1 mM and 7 mM, between 0.1 mM and 6 mM, between 0.1 mM and 4 mM, between 0.1 mM and 3 mM, between 0.1 mM and 2 mM, between 0.1 mM and 1 mM, between 0.1 mM and 0.5 mM, between 0.5 mM and 10 mM, between 0.5 mM and 9 mM, between 0.5 mM and 8 mM, between 0.5 mM and 7 mM, between 0.5 mM and 6 mM, between 0.5 mM and 5 mM, between 0.5 mM and 4 mM, between 0.5 mM and 3 mM, between 0.5 mM and 2 mM, between 0.5 mM and 1 mM, between 1 mM and 10 mM, between 1 mM and 9 mM, between 1 mM and 8 mM, between 1 mM and 7 mM, between 1 mM and 6 mM, between 1 mM and 5 mM, between 1 mM and 3 mM, between 1 mM and 2 mM, between 2 mM and 10 mM, between 2 mM and 9 mM, between 2 mM and 8 mM, between 2 mM and 7 mM, between 2 mM and 6 mM, between 2 mM and 5 mM, between 2 mM and 4 mM, between 3 mM and 10 mM, between 3 mM and 9 mM, between 3 mM and 8 mM, between 3 mM and 7 mM, between 3 mM and 6 mM, between 3 mM and 5 mM, between 3 mM and 4 mM, between 4 mM and 10 mM, between 4 mM and 9 mM, between 4 mM and 8 mM, between 4 mM and 7 mM, between 4 mM and 6 mM, between 4 mM and 5 mM, between 5 mM and 10 mM, between 5 mM and 9 mM, between 5 mM and 8 mM, between 5 mM and 7 mM, between 5 mM and 6 mM, between 6 mM and 10 mM, between 6 mM and 9 mM, between 6 mM and 8 mM, between 6 mM and 7 mM, between 7 mM and 10 mM, between 7 mM and 9 mM, between 7 mM and 8 mM, between 8 mM and 10 mM, between 8 mM and 9 mM, between 8 mM and 9 mM, between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the first electrophoresis medium comprises magnesium ion, calcium ion, copper ion, zinc ion or any combination thereof at a concentration of 1 mM. In some embodiments, the second electrophoresis medium comprises magnesium ion, calcium ion, copper ion, zinc ion or any combination thereof at a concentration sufficient to stabilize the aptamer structure and/or the aptamer-target molecule (e.g., biomolecule) complexes. In some embodiments, the second electrophoresis medium comprises magnesium ion, calcium ion, copper ion, zinc ion or any combination thereof at a concentration of 10 mM or less (e.g., less than 10 mM, less than 9 mM, less than 8 mM, less than 7 mM, less than 6 mM, less than 5 mM, less than 4 mM, less than 3 mM, less than 2 mM, less than 1 mM, or less than 0.5 mM. In some embodiments, the first electrophoresis medium comprises magnesium ion, calcium ion, copper ion, zinc ion or any combination thereof at a concentration of between 0.1 mM and 10 mM, between 0.1 mM and 9 mM, between 0.1 mM and 8 mM, between 0.1 mM and 7 mM, between 0.1 mM and 6 mM, between 0.1 mM and 4 mM, between 0.1 mM and 3 mM, between 0.1 mM and 2 mM, between 0.1 mM and 1 mM, between 0.1 mM and 0.5 mM, between 0.5 mM and 10 mM, between 0.5 mM and 9 mM, between 0.5 mM and 8 mM, between 0.5 mM and 7 mM, between 0.5 mM and 6 mM, between 0.5 mM and 5 mM, between 0.5 mM and 4 mM, between 0.5 mM and 3 mM, between 0.5 mM and 2 mM, between 0.5 mM and 1 mM, between 1 mM and 10 mM, between 1 mM and 9 mM, between 1 mM and 8 mM, between 1 mM and 7 mM, between 1 mM and 6 mM, between 1 mM and 5 mM, between 1 mM and 3 mM, between 1 mM and 2 mM, between 2 mM and 10 mM, between 2 mM and 9 mM, between 2 mM and 8 mM, between 2 mM and 7 mM, between 2 mM and 6 mM, between 2 mM and 5 mM, between 2 mM and 4 mM, between 3 mM and 10 mM, between 3 mM and 9 mM, between 3 mM and 8 mM, between 3 mM and 7 mM, between 3 mM and 6 mM, between 3 mM and 5 mM, between 3 mM and 4 mM, between 4 mM and 10 mM, between 4 mM and 9 mM, between 4 mM and 8 mM, between 4 mM and 7 mM, between 4 mM and 6 mM, between 4 mM and 5 mM, between 5 mM and 10 mM, between 5 mM and 9 mM, between 5 mM and 8 mM, between 5 mM and 7 mM, between 5 mM and 6 mM, between 6 mM and 10 mM, between 6 mM and 9 mM, between 6 mM and 8 mM, between 6 mM and 7 mM, between 7 mM and 10 mM, between 7 mM and 9 mM, between 7 mM and 8 mM, between 8 mM and 10 mM, between 8 mM and 9 mM, between 8 mM and 9 mM, between 0.1 mM and 5 mM, between 0.5 mM and 4.5 mM, between 1 mM and 4 mM, between 2 mM and 3 mM, between 0.5 mM and 2 mM, between 0.6 mM and 1.5 mM, between 0.7 mM and 1.3 mM, between 0.8 mM and 1.2 mM, between 0.9 mM and 1.1 mM, between 0.6 mM and 1.2 mM, between 0.6 mM and 1.2 mM, between 0.7 mM and 1.2 mM, between 0.8 mM and 1.2 mM, between 0.9 mM and 1 mM, between 0.8 mM and 1 mM, between 0.9 mM and 1.5 mM, between 0.9 mM and 1.2 mM, or between 1 mM and 2 mM. In some embodiments, the second electrophoresis medium comprises magnesium ion, calcium ion, copper ion, zinc ion or any combination thereof at a concentration of 1 mM. In some embodiments, the divalent ion is magnesium ion. Magnesium can be added to the first and/or the second electrophoresis medium in any known magnesium salt from such as magnesium chloride, and magnesium sulfate. In some embodiments, the magnesium ion is in the form of magnesium chloride.

In some embodiments, the running buffer of the electrophoresis in the first direction comprises boric acid at a concentration of between 40 mM and 100 mM. In some embodiments, the running buffer of the electrophoresis in the first direction comprises tris(hydroxymethyl)aminomethane at a concentration of between 40 mM and 100 mM. In some embodiments, the running buffer of the electrophoresis in the second direction comprises boric acid at a concentration of between 40 mM and 100 mM. In some embodiments, the running buffer of the electrophoresis in the second direction comprises tris(hydroxymethyl)aminomethane at a concentration of between 40 mM and 100 mM.

In some embodiments, the 2D-electrophoresis is performed at a temperature optimal for migration and separation of the molecules (e.g., aptamer-biomolecule complexes, and unbound aptamer) in the composition. In some embodiments, the electrophoresis in the first direction is performed at a temperature between 8° C. and 22° C., between 9° C. and 21° C., or between 10° C. and 20° C., between 11° C. and 19° C., between 12° C. and 18° C., between 13° C. and 17° C., between 14° C. and 16° C., between 10° C. and 15° C., between 11° C. and 14° C., between 12° C. and 13° C. In some embodiments, the electrophoresis in the first direction is performed at a 8° C., 9° C., 10° C., 11° C., 12° C., 13° C., 14° C., 15° C., 16° C., 17° C., 18° C., 19° C., 20° C., 21° C., or 22° C. In some embodiments, the electrophoresis in the second direction is performed at a temperature between 8° C. and 22° C., between 9° C. and 21° C., or between 10° C. and 20° C., between 11° C. and 19° C., between 12° C. and 18° C., between 13° C. and 17° C., between 14° C. and 16° C., between 10° C. and 15° C., between 11° C. and 14° C., between 12° C. and 13° C. In some embodiments, the electrophoresis in the second direction is performed at 10° C., 11° C., 12° C., 13° C., 14° C., 15° C., 16° C., 17° C., 18° C., 19° C., or 20° C. In some embodiments, the amount of the salt (e.g., sodium chloride and/or magnesium chloride) in the first and the second electrophoresis medium increases the temperature of the electrophoresis medium to a temperature that may interfere with the stability of the aptamer-target molecule complexes. In some embodiments, in order to keep the temperature at the optimal temperature range described herein, the electrophoresis unit is placed in cold water bath filled with ice.

As described herein, in some embodiments, the method further comprises extracting the aptamers capable of binding to one or more target molecules (e.g., biomolecules) from the portion of the second electrophoresis medium that contains the aptamer-biomolecule complexes. In some embodiments, the method further comprising amplifying the plurality of aptamers extracted from the aptamer-target molecule (e.g., biomolecule) complexes to form an aptamer library. In some embodiments, amplification of the aptamers capable of binding to one or more target molecules (e.g., biomolecules) is by polymerase chain reaction (PCR). Polymerase chain reaction (PCR) is a laboratory technique used to amplify DNA sequences by using short DNA sequences called primers. The temperature of the sample is repeatedly raised and lowered to help a DNA replication enzyme copy the target DNA sequences. Non-limiting examples of PCR includes emulsion PCR, Asymmetric PCR, Convective PCR, Dial-out PCR, Digital PCR, Helicase-dependent amplification, Hot start PCR, in silico PCR, Inverse PCR, Ligation-mediated PCR, Miniprimer PCR, Multiplex ligation-dependent probe amplification, Multiplex-PCR, Nanoparticle-Assisted PCR, Nested PCR, Overlap-extension PCR, quantitative PCR, Reverse Complement PCR, Single Specific Primer-PCR, and Solid Phase PCR.

In some embodiments, the plurality of aptamers capable of binding to one or more target molecules (e.g., biomolecules) extracted from the portion of the second electrophoresis medium that contains the aptamer-biomolecule complexes comprises aptamers in different amounts (i.e., a sequence order), e.g., aptamers binding to target molecules with higher affinity are present at a higher level whereas aptamers binding to target molecules with lower affinity are present at a lower level. In some aspects, the present disclosure sought to preserve the sequence order information in the plurality of aptamers capable of binding to one or more target molecules (e.g., biomolecules) after forming the aptamer library by amplification. In some embodiments, any suitable amplification methods known in the art can be employed by the methods described herein provided that the amplification method (e.g., PCR) is capable of preserving the sequence order information in the plurality of aptamers capable of binding to one or more target molecules (e.g., biomolecules) after forming the aptamer library by amplification.

In some embodiments, the present disclosure, at least in part, is based on the discovery that emulsion PCR is capable of preserving the sequence order information in the plurality of aptamers capable of binding to one or more target molecules (e.g., biomolecules) after forming the aptamer library by amplification. The term “emulsion PCR”, as used herein, refers to PCR reaction performed on aqueous droplets emulsified in oil phase of water in oil emulsions. In some embodiments, the aqueous droplets serve as miniaturized “reactors” for each PCR reaction, and are physically separated from each other without exchange of macromolecules, especially the PCR products. In some embodiments, individual DNA molecules are compartmentalized into these distinct reaction droplets, allowing their amplification independent of one another. With emulsion PCR, the formation of unproductive chimeras and other byproducts are avoided, and the overall amplification bias is reduced. (see., e.g., Non Patent Literature 14 and 15).

In some embodiments, enriching a plurality of aptamers is achieved through multiple rounds of selecting for aptamers capable of binding to one or more target molecules (e.g., biomolecules) in a sample (e.g., biological sample). In some embodiments, the steps of the methods described herein are repeated at least once, at least twice, at least three times, at least four times, at least five times, at least six times, at least seven times, at least eight times, at least nine times, at least ten times, at least eleven times, at least twelve times, at least thirteen times, at least fourteen times, at least fifteen times, or more times. In some embodiments, the steps of the methods described herein are repeated four times. In some embodiments, the steps of the methods described herein are repeated nine times. In some embodiments, the aptamer library obtained after amplification (e.g., emulsion PCR) is used as the starting plurality of candidate aptamers for contacting the target molecules (e.g., biomolecules) in the sample (e.g., biological sample) in the next round.

In some embodiments, the method further comprises identifying the sequence of the plurality of aptamers capable of binding to target molecules (e.g., biomolecules) in the sample (e.g., biological sample). Methods of sequencing nucleic acid (e.g., aptamers) are known in the art, which include but are not limited to basic sequencing (e.g., Maxam-Gilbert sequencing), chain-termination sequencing (e.g., Sanger sequencing), large-scale sequencing and de novo sequencing (e.g., shotgun sequencing), next generation sequencing (e.g., Single-molecule real-time sequencing, Ion Torrent sequencing, Pyrosequencing, Sequencing by synthesis (e.g., MiSeq), Combinatorial probe anchor synthesis, Sequencing by ligation (SOLID sequencing), Nanopore Sequencing, GenapSys Sequencing, or Chain termination (Sanger sequencing), long-read sequencing, Short-read sequencing methods (e.g., Massively parallel signature sequencing (MPSS), Polony sequencing, 454 pyrosequencing, Illumina (Solexa) sequencing, DNA nanoball sequencing, Heliscope single molecule sequencing, Microfluidic Systems). In some embodiments, the aptamers are sequenced by next generation sequencing. In some embodiments, the aptamers are sequenced by MiSeq.

In some embodiments, the method further comprises identifying the biomolecules bound to the plurality of aptamers capable of binding to biomolecules in the biological sample. In some embodiments, the biomolecules (e.g., proteins) can be separated from the bound aptamers. Methods of identifying biomolecules are well known to those of ordinary skill in the art. Briefly, methods of identifying biomolecules include, but are not limited to, DNA sequencing (e.g., NGS), northern blot, southern blot, 1D or 2D gel electrophoresis, mass spectrometry, enzyme-linked immunoassay (ELISA), SDS-PAGE, western blot, thin-layer chromatography, high performance liquid chromatography (HPLC), gas chromatography, or any combination thereof. In some embodiments, the biomolecules (e.g., proteins) are identified by mass spectrometry.

In some embodiments, the methods described herein further comprises compiling and storing the sequence information of the aptamer library capable of binding to biomolecules in the biological sample and/or the information of the biomolecules bound to aptamers in the aptamer library to a computer readable format. In some embodiments, the sequence information of the aptamers in the aptamer library and/or the information of the biomolecules bound to the aptamers can be uploaded to a server to form a database. In some embodiments, the database includes information from more than one aptamer library. As the aptamer library is generated, more information of additional different aptamer libraries can be added to the database (e.g., the database can include an aptamer library information for mouse serum, an aptamer library information for mouse CSF, an aptamer library information for human serum, an aptamer library information for human CSF, an aptamer library information for a cancer tissue, etc.). Once the aptamer library information is identified and added to the database, such information can be readily downloaded and used without the need to further perform the steps in the methods described herein. In some embodiments, the aptamer database is stored in a server. In some embodiments, the server that stores the aptamer database further provides means for downloading the aptamer library information stored therein. In some embodiments, the server that stores the aptamer database further provides means for exporting the aptamer library information for comparison to another aptamer library on the server.

### II. Methods of Using the Information of Aptamer Library

In some aspects, the present disclosure also provides a method for profiling a target aptamer library for a target biological sample, the method comprising: (i) acquiring the information of the target aptamer library for the target biological sample from a first aptamer database, (ii) acquiring the information of a reference aptamer library for a reference biological sample from a second aptamer database, (iii) comparing the information of the target aptamer library for the target biological sample and the reference aptamer library for the reference biological sample thereby profiling the target aptamer library. It will be recognized by those of skill in the art that the terms “acquiring”, “providing”, “retrieving”, or “procuring” are used interchangeably, and refer to steps comprising accessing an aptamer database, locating information (e.g., aptamer sequence identities, aptamer modifications, aptamer relative abundance, aptamer purity, etc.) corresponding to an aptamer library in order to provide that information in a subsequent step (e.g., step (iii) wherein the information of the target aptamer library and the reference aptamer library are compared). The term “profiling a target aptamer library”, as used herein, refers to analyzing biological information or characteristics of a target subject based on information of a target aptamer library for a target biological sample and a reference aptamer library for a reference biological sample (e.g., sequence information including but not limited to sequence frequency or quantity of aptamers in the aptamer library capable of binding to biomolecules in the biological sample).

“A method for profiling a target aptamer library” can provide a method for determining the state of physical condition in a target subject or diagnosing diseases in a target subject.

The diagnosis of diseases can, for example, be based on differences between information of an aptamer library for a biological sample from a healthy subject and information of an aptamer library for a biological sample from a subject having or suspected of having a disease. When the information of the target aptamer library for the target biological sample is compared with the information of the reference aptamer library for the reference biological sample from the subject having or suspected of having the disease, the target subject is diagnosed with the disease or the risk of the disease if it has at least one feature representing characteristic information of the reference aptamer library (e.g., high/low sequence frequency or quantity of particular aptamer(s)).

Diseases can be diagnosed with by comparing the information of the target aptamer library for the target biological sample with the information of the reference aptamer library for the reference biological sample from the healthy subject. If the information of the target aptamer library for the target biological sample is essentially the same as the information of the reference aptamer library for the reference biological sample from the healthy subject, the target subject is considered to be healthy. If the information of the target aptamer library for the target biological sample shows any difference relative to the information of the reference aptamer library for the reference biological sample from the healthy subject, it can be determined that the target subject has any disease or any risk of disease.

Alternatively, or in addition, the information of the target aptamer library for the target biological sample may be compared with the information of the reference aptamer library for the reference biological sample from a subject independently diagnosed with the disease or the risk of the disease. In this case, the target subject is diagnosed with the disease or the risk of the disease if it has at least one feature representing characteristic information of the reference aptamer library (e.g., high/low sequence frequency or quantity of particular aptamer(s)).

“A method for profiling a target aptamer library” may comprise identify biomolecules bound to aptamers in the target aptamer library or the reference aptamer library that show the difference between the information of the target aptamer library for the target biological sample and the information of the reference aptamer library for the reference biological sample.

In some aspects, the present disclosure also provides a non-transitory computer-readable medium having stored thereon instructions that, when executed by at least one processor, cause the at least one processor to perform a method for profiling a target aptamer library for a target biological sample, the method comprising: (i) acquiring the information of the target aptamer library for the target biological sample from a first aptamer database, (ii) acquiring the information of a reference aptamer library for a reference biological sample from a second aptamer database, (iii) comparing the information of the target aptamer library for the target biological sample and the reference aptamer library for the reference biological sample thereby profiling the target aptamer library.

In some embodiments, the first aptamer database is generated by the methods (e.g., methods of enriching a plurality of aptamers capable of binding to one or more biomolecules in a biological sample) described herein. In some embodiments, the second aptamer database is generated by the methods (e.g., methods of enriching a plurality of aptamers capable of binding to one or more biomolecules in a biological sample) described herein. In some embodiments, the first aptamer database and the second aptamer database are generated by the methods (e.g., methods of enriching a plurality of aptamers capable of binding to one or more biomolecules in a biological sample) described herein. In some embodiments, the first aptamer database and the second aptamer database are the same. In some embodiments, the first aptamer database and the second aptamer database are different.

In some embodiments, the aptamer library information is initially obtained by the methods described herein (e.g., by BIOLLET) and stored in an aptamer database. In some embodiments, acquiring the information of an aptamer library (e.g., a target aptamer library and/or a reference aptamer library) from an aptamer database does not require performing the steps of obtaining the aptamer library information (e.g., by BIOLLET) again.

In some embodiments, the method further comprises downloading the information of the target aptamer library for the target biological sample from the aptamer database to a user computer or extracting the information of the target aptamer library for the target biological sample from the aptamer database to a server where the aptamer database is stored. In some embodiments, the method further comprises downloading the information of the reference aptamer library for the reference biological sample from the aptamer database to a user computer or extracting the information of the reference aptamer library for the reference biological sample from the aptamer database to a server where the aptamer database is stored.

In some embodiments, the method further comprises determining the difference between the information of the target aptamer library and the reference aptamer library.

The target aptamer library and/or the reference aptamer library can be selected based on the purpose of the comparison.

In some embodiments, the target biological sample is a biological sample from a subject of the same genetic background as the reference biological sample. For example, the target biological sample is serum from C57BL/6 mouse, and the reference biological sample is serum from another C57BL/6 mouse. In some embodiments, the target biological sample is serum from a human subject of an ethnicity, and the reference biological sample is serum from a human subject of the same ethnicity. By comparing the target aptamer library and the reference aptamer library, the differences in serum molecules (e.g., proteins) can be identified and the difference may suggest a factor may have affected serum molecule (e.g., protein). Such factor may include, but not limited to environmental factors, dietary factors, epigenetic modifications, disease conditions, age, or gender).

In some embodiments, the target biological sample is a biological sample from a subject of different genetic background as the reference biological sample. For example, the target biological sample is serum from C57BL/6 mouse, and the reference biological sample is serum from a BALB/c mouse. In some embodiments, the target biological sample is serum from a human subject of an ethnicity, and the reference biological sample is serum from a human subject of a different ethnicity. By comparing the target aptamer library and the reference aptamer library, the differences in serum molecules (e.g., proteins) can be identified and the difference may suggest a certain genetic background is prone to or at risk of having a certain disease.

In some embodiments, the target biological sample and the reference biological sample are from the same subject but obtained at different times. For example, the target biological sample is serum from a human subject prior to a treatment, and the reference biological sample is serum from the same human subject after a treatment. By comparing the target aptamer library and the reference aptamer library, the differences in serum molecules (e.g., proteins) can be identified and the difference may suggest the efficacy of the treatment. In other embodiments, the target biological sample is serum from a human subject when the subject has or is suspected of having a disease, and the reference biological sample is serum from the same human subject when the subject is healthy. By comparing the target aptamer library and the reference aptamer library, the differences in serum molecules (e.g., proteins) can be identified and the difference may suggest that the subject has the suspected disease or the risk of the suspected disease.

In some embodiments, the target biological sample is obtained from a subject having or suspected of having a disease, and the reference biological sample is obtained from a healthy individual. For example, the target biological sample is serum from a human subject having or suspected of having a disease, and the reference biological sample is serum from a healthy human subject. By comparing the target aptamer library and the reference aptamer library, the differences in serum molecules (e.g., proteins) can be identified and the difference may suggest that the target subject has the suspected disease or the risk of the suspected disease.

In some embodiments, the target biological sample is obtained from a subject having or suspected of having a disease, and the reference biological sample is obtained from a subject having or suspected of having certain disease. For example, the target biological sample is serum from a human subject having or suspected of having a disease, and the reference biological sample is serum from a subject having or suspected of having certain disease. By comparing the target aptamer library and the reference aptamer library, it is possible to diagnose the human subject's disease.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an”, as used herein in the specification and in the claims, unless clearly indicated to the contrary, should be understood to mean “at least one”.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”. “Consisting essentially of”, when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B”, or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

### EXAMPLES

### Materials and Methods

### Preparation of Agarose Gel for 1D- and 2D-Electrophoresis

The agarose gel for 1D- and 2D-electrophroesis was prepared as follows. Agarose powder was mixed at a concentration of 1.0-1.2 wt % in 1×TB buffer (80 mM tris base, 80 mM boric acid) including 100-200 mM NaCl concentration which is dependent on selection condition. The mixture was heated by microwave to completely dissolve agarose powder. One molar magnesium chloride solution was added at a concentration of 1 mM in the agarose solution after cooling down to around 60° C. and mixed thoroughly. Eleven milliliters of the solution were poured into a gel tray (55 mm (W)×60 mm (L)) with a well comb in place. The number of well is 3-4 wells (10 mm (W)×1 mm (L)) for 1D-electrophoresis and 1 well (45 mm (W)×2 mm (L)) for 2D-electrophoresis. The thickness of agarose gel is adjusted to be relatively thin to decrease heat generated from high-salt agarose gel during electrophoresis. The gel was placed at room temperature for at least 3 hours, but less than 6 hours, before use.

### Preparation of DNA Library for Binding Reaction

A single-stranded DNA (ssDNA) library with a random region of 43 nucleotides and TYE665 fluorophore at the 5′-end was employed for selections. The ssDNA library was diluted in the selection buffer (PBS with 0.005% nonidet-P40 and 1 mM magnesium chloride) at a certain concentration ranging from 100 nM to 5000 nM depending on selection condition. The ssDNA library was denatured at 95° C. for 3 min then slowly cooled down to room temperature over 30 min to form stable ssDNA structure. The renatured ssDNA library solution was mixed with an appropriately diluted biofluid sample solution at 10 μL reaction volume. Dilution range of biofluid sample depends on type of biofluid; for example, 2 to 200 times dilution value for serum sample and 1 to 2 times dilution value for cerebrospinal fluid (CSF) sample are employed. The biofluid sample was mixed with competitors, such as salmon sperm DNA and any unrelated DNA oligos, in advance to mixing with the ssDNA library solution. Dextran sulfate was also employed to reduce charge-dependent non-specific interaction of ssDNA with biomolecules. The mixture was incubated at room temperature for 10 min, and then 2 μL of 6× loading buffer comprising 36% glycerol and 6 mM magnesium chloride was added to the reaction mixture quickly followed by loading the sample on the agarose gel.

### 1D-Electrophoresis Separation

An electrophoresis unit, such as Mupid2, was filled with fresh 1×TB buffer. The gel for 1D-electrophoresis prepared above was placed in the gel box and the electrophoresis unit was placed in a cold water bath filled with crushed ice to keep the buffer temperature between 10-20° C. during electrophoresis due to the use of high-salt agarose gel. The gel was pre-run at 100 V for 10 min before sample run. The sample mixture was carefully loaded on the well of the gel. First-dimensional electrophoresis was performed at 100 V for 55-60 min in the dark place. The running time is dependent on selection round. After the run, the gel was taken from the gel box and visualized by ChemiDoc MP Imaging System (Bio-Rad). The entire region of aptamer-biomolecule complexes that are generally present above free aptamer region was excised for 2D-electrophoresis. The size of excised gel was adjusted to the well of 2D-electrophoresis gel.

### 2D-Electrophoresis Separation

The other electrophoresis unit was filled with fresh 1×TB buffer and the gel for 2D-electrophoresis was placed in the gel box. As with 1D-electrophoresis, the equipment was placed in a cold water bath as with 1D-electrophoresis and the gel was pre-run at 100 V for 10 min before performing 2D-electrophoresis. This pre-run step was performed during running 1D-electrophoresis in order to start 2D-electrophoresis right after gel excision. After the pre-run, the gel was removed from the gel box and the excised gel was carefully inserted into the well. The gel was set back to the gel box and started 2D-electrophoresis at 100 V for 55-60 min in the dark place. The running time is dependent on selection round. After the run, the gel was taken from the gel box for visualization by ChemiDoc MP Imaging System (Bio-Rad) and only diagonal region formed by aptamer-biomolecule complexes was excised. The excised gel piece was placed in a microtube.

DNA Extraction from Agarose Gel

The excised gel piece was melted at 95° C. for 3 min. The melted agarose solution was distributed into several tubes with 110 μL aliquots. The tubes were incubated at 55° C. for 1 min. Thermostable β-agarase was added to each tube by 2 μL and the tubes were incubated at 55° C. for 15 min to enzymatically digest agarose. The ssDNA library was recovered by Oligo Clean and Concentrator Kits (Zymo Research), following manufacturer's instructions. The recovered ssDNA was eluted with 30 μL of deionized water.

PCR Amplification of Recovered ssDNA Library

The recovered ssDNA was subjected to PCR amplification in a two-step process. In the first step, test PCR reactions comprising different numbers of sequential PCR cycles were performed with a small volume. Note that all PCR amplifications were performed as an emulsion PCR procedure in which 100 μL of PCR solution was mixed with 250 μL of emulsion oil (4.5% Span 80, 0.4% Tween 80, 0.05% Triton-X 100, and 95.05% Mineral Oil) and the solution was vigorously mixed by magnetic stir bar until completely mixed. The PCR products at defined cycles were recovered by chloroform extraction and analyzed by gel electrophoresis (6% polyacrylamide with 0.5×TBE buffer) to determine a proper PCR cycle that can provide a clear single band for DNA products without any concatemers and truncations. The remaining PCR sample was amplified by employing the determined PCR cycle. The PCR products were recovered by chloroform extraction and subjected to purification by Oligo Clean and Concentrator Kits. The amplified DNA was eluted with 10 μL of deionized water and quantified by Qubit 4 Fluorometer (Invitrogen). This PCR amplification step was repeated until enough amount of DNA was obtained.

### Aptamer Library Generation by Primer Extension

The amplified DNA was diluted to the concentration of 1 ng/μL to prepare template DNA sample for aptamer library generation by primer extension. Two microliters of the solution were used for 100 μL of each PCR amplification. In this PCR amplification step, 5′-biotinylated antisense strand primer was employed, and the number of PCR cycle was fixed to 8 cycles. The PCR products were recovered by chloroform extraction followed by Oligo Clean and Concentrator Kits purification. The purified DNA was subjected to primer extension by using 5′-TYE665 fluorophore-labeled primer. The products were directly immobilized on streptavidin agarose beads filled with PBSN buffer (PBS with 0.005% nonidet-P40). The beads were incubated at 16° C. with shake at 1500 rpm every 2 min for 15 min. The beads were washed 3 times with PBS and filled with 40 μL of 20 mM NaOH solution for denaturation of the product DNA to dissociate 5′-TYE665-labeled aptamers. The beads solution was incubated at 37° C. with shake at 1500 rpm for 1 min. The solution was centrifuged by a benchtop mini centrifuge for 30 sec. The supernatant was transferred to a new tube. The denaturation step was performed once more, and the supernatants were combined in the same tube. The collected supernatant was quenched by 80 mM HCl to adjust the solution pH around 7.0-8.0 and purified by Oligo Clean and Concentrator Kits. The aptamer library was eluted with 10 μL of deionized water and quantified by NanoDrop. The aptamer library was used for the next cycle of selection.

### Sequencing Analysis of Aptamer Library

The PCR products obtained at each round of selection were PCR amplified in a 2-step process to prepare for next generation sequencing analysis by MiSeq (illumina). Adapter sequence primer set was used in the first step followed by using index sequence primer set in the second step. Thermal cycling was performed as in the manufacturer's instruction. The PCR products were purified by NucleoSpin Gel and PCR Clean-up (Macherey Nagel) and quantified by Qubit 4 fluorometer for the first step and Bioanalyzer for the second step. The products were analyzed by MiSeq following manufacturer's instructions. All sequencing data for each round were generated as FASTQ files. After extracting aptamer domain by trimming 5′- and 3′-primer regions, all sequences were used for frequency analysis to investigate generation of aptamers during selections.

### Comparative Analysis of Information of Aptamer Libraries

The method according to the present disclosure was applied for the comparative analysis of information of aptamer libraries. An aptamer library was selected based on the results for sequence analysis of aptamer libraries. The aptamer library was renatured to form stable structures and then mixed with samples. The condition was the same as the one that prepared for the aptamer library. The plurality of aptamer-biomolecule complexes were purified by the procedure of 1D-electrophoresis. The recovered ssDNAs from the gels were amplified with fixed PCR cycles and directly analyzed by next generation sequencing without 2D-electrophoresis. The information of the aptamer library was obtained by calculating % frequency of each sequence in the total number of reads for each sample. The % frequency information (the information of the aptamer library) was compared among various reference biological samples to assess changes in the states of biomolecules.

### Example 1: Aptamer Library Generation for Mouse Serum

The method according to the present disclosure was applied for the generation of aptamer library targeting mouse serum sample.

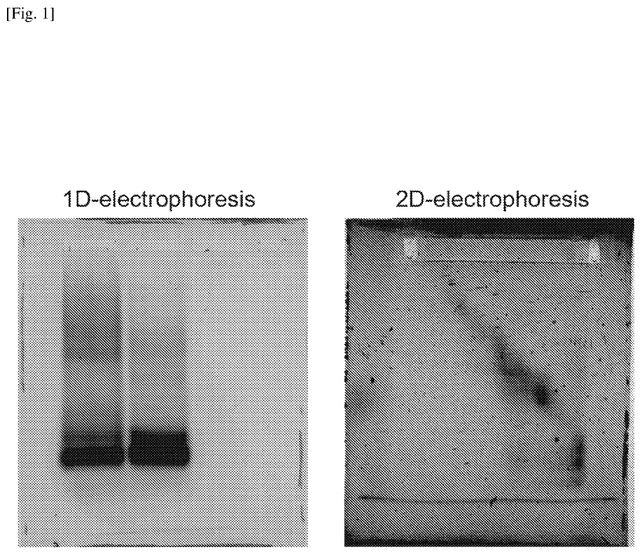

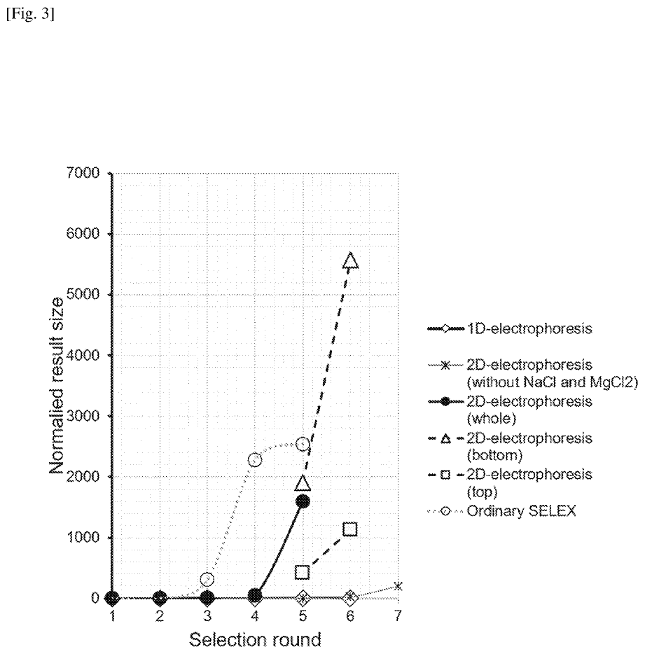

Selection was performed for 5 selection rounds, using a completely random DNA library. The starting library contained a random region of 43 nucleotides composed of adenine, guanine, cytosine, and 5-tryptamino-uracil instead of thymine. Representative imaging data for 1D- and 2D-electorophoresis were shown in FIG. 1. Specific indication that selection was successful is the observation that the number of certain sequences increase over selection rounds. The other indication is the observation that different sequence variations appear in different regions of the gel after electrophoresis. For this purpose, 5th and 6th rounds were additionally performed with modified procedures in which the gel after 1D-electrophoresis was separated into 2 parts, top and bottom as shown in FIG. 2, for the analysis of specific sequence evolution. In next generation sequencing analysis, averaged sequencing reads were 50,933. The library growth curve was shown in FIG. 3 in which the result size indicates number of sequences with more than 3 counts. For comparison, growth curves for ordinary SELEX data and 1D-electrophoresis-based biofluid SELEX data are also shown in FIG. 3. The top twenty sequences in terms of frequency in the library from whole gel are listed below in table 1. The top twenty sequences from different regions of the gel, bottom and top regions, are listed below in table 2 and table 3, respectively.

TABLE 1

| SEQ. ID | | Frequency (%) |
|---|---|---|
| 1 | CCAtACAGAttAACAAAAACAttACttttttAAAGtGGACAAAAtCC | 0.09 |
| 2 | CCAGAACGttGtAGCCAtAtGCGCACGACGtAACGACCLAAGCAACC | 0.05 |
| 3 | CCGttGtAGCCAtAtGCGGAACAAGAAttAGGGAtCGCAtCGCAACC | 0.04 |
| 4 | CCtAACCCAAAAtCCCACAtGAtAttGtGtCCAtACAtCCAtCAtCC | 0.04 |
| 5 | CCtAAGAAACGGAAtttAttttCtCttAAAGtGGACGAGACGtGACC | 0.03 |
| 6 | CCACGAAGGAAtACACttGtttttAtGttAGAGGACtGGAtGAtCCC | 0.03 |
| 7 | CCtAGAGGGAtGtGtAGtGCtCGGAGCAtCCCtGAAAtGCAtCAtCC | 0.03 |
| 8 | CCtAAAACGAAGAtGACtttAttttttCAGtGGACGGAtACGGAGCC | 0.03 |
| 9 | CCGtACACAtttGGCtttttAAAtGAtttCAtAAGGGACtGAtAACC | 0.03 |
| 10 | CCAtAAAGACCGCCGGGACGGGCGACtGttGtAGCtAtAtGCAGCCC | 0.03 |
| 11 | CCCGCCCAACtAAttttAttCttCtAAGtGGAtGAAAGAtCCGCACC | 0.03 |
| 12 | CCAACGAACGAtCAttAGGttGAAGCtAtAtGCCAGGttCAtGACCC | 0.03 |
| 13 | CCCAAAAACAGCAttCtttCttttAtAAAGtGGACCAtCAAAGCtCC | 0.03 |
| 14 | CCACCAtCAtAtAAAAttttGAtttAAAGtGGACtttACAGGCAACC | 0.03 |
| 15 | CCAAGACGACAtGAtAttttGCttAtAtttGAAAGtGGACAtCAtCC | 0.02 |
| 16 | CCtGttGtGttttttAAAGtGGGGtGAAtAAGCAAAAACtCttAtCC | 0.02 |
| 17 | CCCAAACCGttGtAGCAAtAtGCGCCttCCtGCCCtAAtAGACCACC | 0.02 |
| 18 | CCttAAGtACGtCAAttGAtCGttttttAGtGGACtGACtCtAAACC | 0.02 |
| 19 | CCAAACGACAGAAACAGttACttttttAtAAGGACGAtAtCGAtCC | 0.02 |
| 20 | CCtAAGCAGtAtGAACCtttAtttttAtGGGGCGAGACGAtCtGtCC | 0.02 |

Table 1. List of top 20 aptamer sequences obtained from whole gel analysis is shown. Small letter “t” means 5-tryptamino-uracil.

TABLE 2

| | | Frequency (%) | | |
|---|---|---|---|---|
| SEQ. ID | | Whole | Bottom | Top |
| 21 | CCATACAGAttAACAAAAACAttACttttttAAAGtGGACAAAAtCC | 0.09 | 0.33 | 0.01 |
| 22 | CCAGAACGttGtAGCCAtAtGCGCACGACGLAACGACCLAAGCAACC | 0.05 | 0.29 | 0.01 |
| 23 | CCGttGtAGCCAtAtGCGGAACAAGAAttAGGGAtCGCAtCGCAACC | 0.04 | 0.21 | 0.01 |
| 24 | CCAACGAACGAtCAttAGGttGAAGCtAtAtGCCAGGttCAtGACCC | 0.03 | 0.16 | 0.00 |
| 25 | CCtAACCCAAAAtCCCACAtGAtAttGtGtCCAtACAtCCAtCAtCC | 0.04 | 0.13 | 0.00 |
| 26 | CCAtAAAGACCGCCGGGACGGGCGACtGttGtAGCtAtAtGCAGCCC | 0.03 | 0.11 | 0.00 |
| 27 | CCtAAAACGAAGAtGACtttAttttttCAGtGGACGGALACGGAGCC | 0.03 | 0.10 | 0.00 |
| 28 | CCtGGttGtAGCAAtAtGCCCCGGAGtAtCGGtCGtCCAAAAGAGCC | 0.01 | 0.10 | 0.00 |
| 29 | CCtAGAGGGAtGtGtAGtGCtCGGAGCAtCCCtGAAAtGCAtCAtCC | 0.03 | 0.10 | 0.00 |
| 30 | CCCCGAGAGAAGGttGtAGCAAtAtGCCCGAGGGGAtCCGAACCtCC | 0.02 | 0.08 | 0.00 |
| 31 | CCAAGACGACAtGAtAttttGCttAtAtttGAAAGtGGACAtCAtCC | 0.02 | 0.08 | 0.00 |
| 32 | CCCAAAAACAGCAttCtttCttttAtAAAGtGGACCAtCAAAGCtCC | 0.03 | 0.07 | 0.00 |
| 33 | CCtAAGAAACGGAAtttAttttCtCttAAAGtGGACGAGACGEGACC | 0.03 | 0.06 | 0.00 |
| 34 | CCCAGGACAAAAGAtACCAGAAGAtttttCttAAGtGGACGGAtACC | 0.02 | 0.06 | 0.00 |
| 35 | CCCGCCCAACtAAttttAttCttCtAAGtGGAtGAAAGAtCCGCACC | 0.03 | 0.06 | 0.01 |
| 36 | CCtGttGtGttttttAAAGtGGGGtGAAtAAGCAAAAACtCttAtCC | 0.02 | 0.06 | 0.01 |
| 37 | CCCCAtAAGCGtGtCtGCGtCAttACtAGtACtACACCtGtCAGCCC | 0.01 | 0.06 | 0.00 |
| 38 | CCGtCAACCCAAtCAACAttttGtGttttAttGtGGACtGACACACC | 0.02 | 0.06 | 0,00 |
| 39 | CCLAAtGALAAtCAAAACAGCttGtAGCtAtAtGGCtCAGtCtAtCC | 0.01 | 0.06 | 0.00 |
| 40 | CCttGtCCAAtAtACtCAttACtCGtACtAGtCCtCAGCtGACtGCC | 0.01 | 0.06 | 0.00 |

Table 2. List of top 20 aptamer sequences obtained from bottom gel part indicated in FIG. 2 is shown. The frequency of these aptamers is compared with whole gel data and top gel data to investigate specificity. Small letter “t” indicate 5-tryptamino-uracil.

TABLE 3

| | | Frequency (%) | | |
|---|---|---|---|---|
| SEQ. ID | Sequence | Whole | Bottom | Top |
| 41 | CCAttttACtCtCAtAtACGAAAGGtACCLAtAGAAtCAttAGttCC | 0.01 | 0.01 | 7.05 |
| 42 | CCCtGCtttGGAGtGGAtACtAAtCGAtttAGGAAtGCttAtCtCC | 0.00 | 0.00 | 3.27 |
| 43 | CCAAACAttCtAAtttGAAttGtGAAGCAtttCCtCtCCGtGtCACC | 0.00 | 0.00 | 3.11 |
| 44 | CCtCAAtACtAtCAGAACGAttGAtAtGtAtttAtAACAAttAAACC | 0.00 | 0.00 | 2.54 |
| 45 | CCLACtGGtGGGAAttCtAGGGtACttAAtAAGtCCCtCAtAttGCC | 0.01 | 0.00 | 2.37 |
| 46 | CCAtACtAGGtGttGtGCAttACtttAAtAGCGCCtAtACAAAGtCC | 0.00 | 0.00 | 2.06 |
| 47 | CCACtACttttAAAAAGGCACAtAGtGACGtAtttAAACAGtCGACC | 0.00 | 0.00 | 1.42 |
| 48 | CCAttACCGtCAAtAttGAtGAAtGtCAtAttGACttGttGGtCGCC | 0.01 | 0.00 | 1.31 |
| 49 | CCGttAAtAAtCCCGAtACtAttttAGtACAtttGGCCAAAtGtACC | 0.00 | 0.00 | 1.30 |
| 50 | CCAAAAtAACAtCtGCCCtttACAAGtAACAGAtGtAtAtGtCGACC | 0.00 | 0.00 | 1.16 |
| 51 | CCCCAttCAGACAACCCttGGtGttCAAttCAttGCAACGAGtGtCC | 0.00 | 0.00 | 1.12 |
| 52 | CCAtCACAtCtAttGAGGtttACCCttGACGCGtCAtAAACttCACC | 0.01 | 0.00 | 1.09 |
| 53 | CCCCCtCAGtGttAGGAGGtAGAAtACtCCtCAACACAtCtCGtCCC | 0.00 | 0.00 | 1.02 |
| 54 | CCttCAGtCCtCAtAttAGGtAGACttCtGGtCCtAAtAtAttCACC | 0.01 | 0.00 | 1.02 |
| 55 | CCCAttGttAGAAttGttAAAttCCtGACAGtCGtCGAtAAtGttCC | 0.00 | 0.00 | 0.91 |
| 56 | CCtGtGtAtGGCtCGttGCtGGAtCtGAtAGGtGCttCttCCACCCC | 0.00 | 0.00 | 0.78 |
| 57 | CCAAtGAtACtttACtGGAttAtGtAAAACGCGCCtAtACAAAGtCC | 0.00 | 0.00 | 0.74 |
| 58 | CCAAtAtGCGAtGAtGAAtGtCAGAGCAGCtCAttGtGtAtttGACC | 0.00 | 0.00 | 0.71 |
| 59 | CCtACCttAACttCGtAtGtAAACttGAtGGAtGtCAtttACACtCC | 0.00 | 0.00 | 0.57 |
| 60 | CCtGCttGttAACttGAAAAGttACAACtGtAAACttGCttACtGCC | 0.00 | 0.00 | 0.57 |

Table 3. List of top 20 aptamer sequences obtained from top gel part indicated in FIG. 2 is shown. The frequency of these aptamers is compared with whole gel data and bottom gel data to investigate specificity. Small letter “t” indicates 5-tryptamino-uracil.

### Example 2: Comparative Analysis of Information of Aptamer Libraries for Mouse Serum

The method according to the present disclosure was applied for the comparative analysis of information of aptamer libraries for mouse serum samples from the same or different strains.

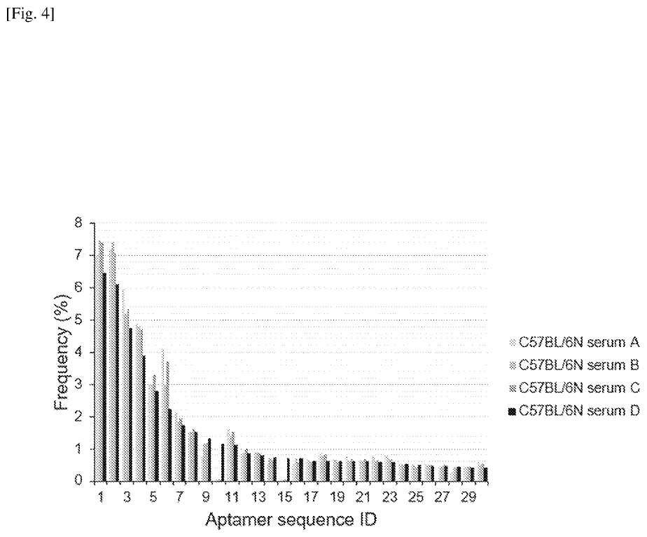

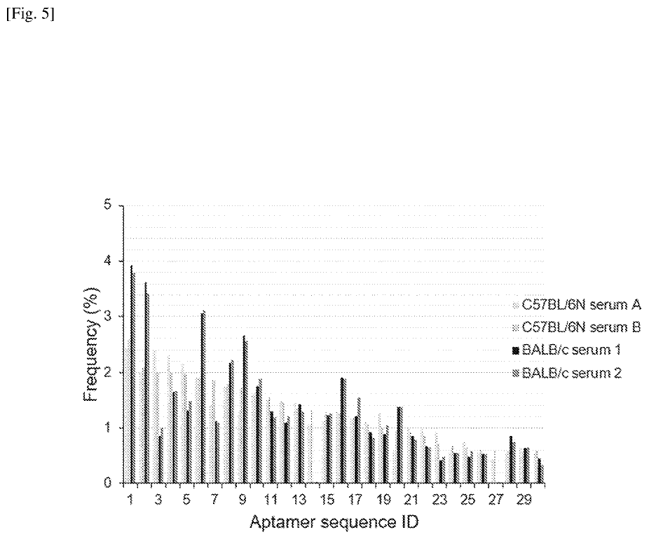

Serum samples were prepared from C57BL/6N (B6/N) and BALB/c mouse strains. One of C57BL/6N mouse serum was employed to generate aptamer libraries. An aptamer library was selected based on sequencing results after 5 rounds of selection. A gradual slope of sequence frequencies with around 1-2% top sequence frequency was the basic selection criteria. The aptamer library was interacted with several serum samples from C57BL/6N or BALB/c strains and the aptamer-protein complexes were purified by 1D-electrophoresis followed by next generation sequencing analysis using MiSeq (illumina). Results from comparative analysis of information of aptamer libraries between the same mouse strain samples (C57BL/6N v.s. C57BL/6N) or different mouse strain samples (C57BL/6N v.s. BALB/c) were shown in FIG. 4 and FIG. 5, respectively. Note that different aptamer library was used for each comparative analysis.

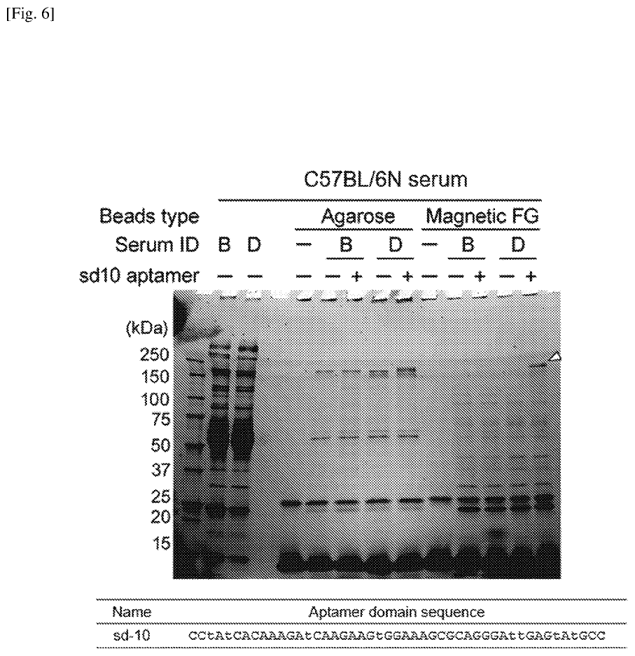

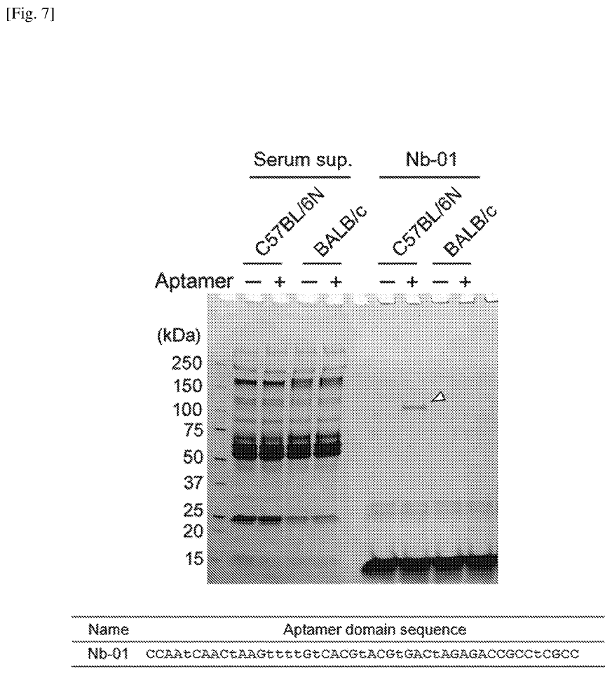

Some aptamers that showed sample specific patterns were selected to test the target specificity by aptamer-based precipitation assay. One was sd-10 aptamer that showed high specificity toward only serum D in the same strain analysis, and the other was Nb-01 aptamer which showed specificity to only C57BL/6N serum acquired from the different mouse strain analysis. Both aptamers were modified with biotin at the 5′-end to precipitate target molecules. These aptamers were interacted with serum samples followed by a pull-down assay by using streptavidin beads (agarose beads or magnetic FG beads). After sufficient wash, the beads were analyzed by non-denaturing stain-free SDS-PAGE. The gels were visualized by ChemiDoc MP Imaging System (Bio-Rad) and shown in FIG. 6 and FIG. 7 for the pull-down results of sd-10 aptamer and Nb-01 aptamer, respectively.

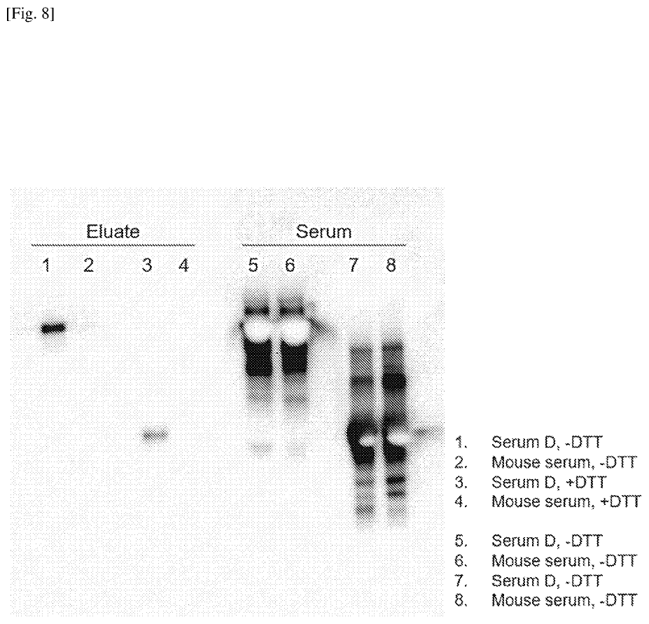

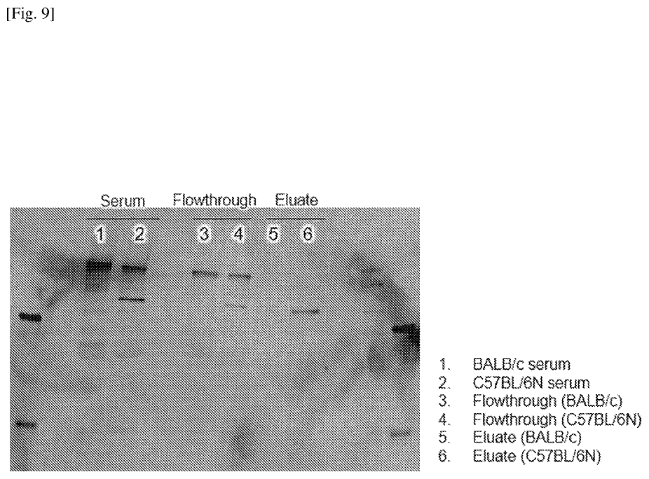

Target proteins of sd-10 and Nb-01 aptamers were identified to further confirm the molecular specificity of these aptamers. Target bands were excised and used for MS analysis. Results indicated that sd-10 aptamer recognized mouse IgG and Nb-01 aptamer recognized GPLD-1. Western blotting analysis by using mouse IgG antibody (FIG. 8) and GPLD-1 antibody (FIG. 9) confirmed results of MS analysis. As shown in FIG. 8, sd-10 aptamer recognized only small portions of total antibody in serum, indicating specific interaction to a certain type of antibody that was differentially expressed.

### Example 3: Comparative Analysis of Information of Aptamer Libraries for Human Cerebrospinal Fluid Samples

The method according to the present disclosure was applied for the comparative analysis of information of aptamer libraries for human cerebrospinal fluid (CSF) samples.

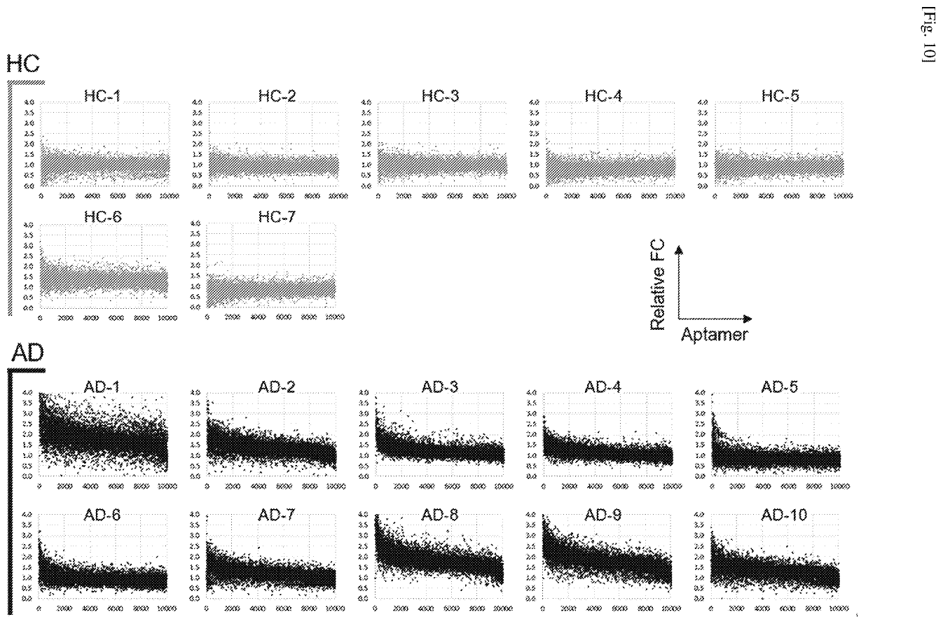

CSF samples were purchased from PrecisionMed, LLC. Mixture of five CSF samples derived from 5 AD patients were employed to generate aptamer libraries targeting human CSF samples. An aptamer library was selected based on the same procedures as in the Example 2. The aptamer library was interacted with each CSF sample derived from 7 healthy volunteers (HC) and 10 AD patients. Aptamer-protein complexes were purified by 1D-electrophoresis followed by next generation sequencing analysis using HiSeq X (illumina). Each sequence frequency was obtained as profiling score. Averaged profiling score of HC was employed as a baseline and a fold change (FC) of each sequence from the baseline was calculated and plotted as in FIG. 10. The comparative analysis of information of aptamer libraries showed different patterns of aptamer reactivity toward HC and AD CSF samples. Some aptamers that show the difference between the information of the aptamer library for the AD CSF sample and the aptamer library for the HC CSF sample can be selected, and the biomolecules bound to the aptamers in the aptamer library for the AD CSF sample or the aptamer library for the HC CSF sample can be identified by performing as described in Example 2.

1. A method for enriching a plurality of aptamers capable of binding to one or more biomolecules in a biological sample, the method comprising:
(i) contacting a plurality of candidate aptamers with a biological sample to form a composition comprising a plurality of aptamer-biomolecule complexes;
(ii) subjecting the composition to electrophoresis in a first electrophoresis medium in a first direction to obtain a portion of the first electrophoresis medium that comprises the plurality of aptamer-biomolecule complexes;
(iii) subjecting the portion of the first electrophoresis medium to electrophoresis in a second electrophoresis medium in a second direction to obtain a portion of the second electrophoresis medium that comprises the plurality of aptamer-biomolecule complexes; and
(iv) extracting the plurality of aptamers capable of binding to biomolecules in the biological sample from the plurality of aptamer-biomolecule complexes.

2. The method of claim 1, further comprising (v) amplifying the plurality of aptamers capable of binding to biomolecules in the biological samples to form an aptamer library.

3. The method of claim 2, wherein the plurality of aptamers capable of binding to biomolecules in the biological samples are amplified by polymerase chain reaction (PCR).

4. The method of claim 3, wherein the PCR is emulsion PCR.

5. The method of claim 1, wherein step (ii) further comprises excising the portion of the first electrophoresis medium comprising the plurality of aptamer-biomolecule complexes from the rest of the first electrophoresis medium.

6. The method of claim 5, wherein the portion of the first electrophoresis medium is fitted to a well in the second electrophoresis medium for performing the electrophoresis in the second direction.

7.-17. (canceled)

18. The method of claim 1, wherein the biological sample is not denatured.

19.-32. (canceled)

33. The method of claim 1, further comprising identifying the biomolecules bound to the plurality of aptamers capable of binding to biomolecules in the biological sample.

34. (canceled)

35. An aptamer database comprising information of one or more aptamer libraries generated by the method of claim 1, wherein each aptamer library comprises the aptamers capable of binding to biomolecules in a biological sample.

36. A method for profiling a target aptamer library for a target biological sample, the method comprising:
(i) acquiring the information of the target aptamer library for the target biological sample from a first aptamer database,
(ii) acquiring the information of a reference aptamer library for a reference biological sample from a second aptamer database, and
(iii) comparing the information of the target aptamer library for the target biological sample and the reference aptamer library for the reference biological sample thereby profiling the target aptamer library,
wherein the first aptamer database in step (i) is the aptamer database of claim 35.

37.-41. (canceled)

42. The method of claim 36, further comprising (iv) determining the difference between the information of the target aptamer library and the reference aptamer library.

43.-45. (canceled)

46. The method of claim 36, wherein the target biological sample is from a target subject having or suspected of having a disease.

47. The method of claim 46, wherein the reference biological sample is from a healthy subject.

48. The method of claim 46, wherein the reference biological sample is from a subject having or suspected of having a disease.

49. The method of claim 46, wherein the comparing step results in determining whether the target subject has the disease or the risk of the disease.

50. The method of claim 36, wherein the target biological sample is a diseased sample.

51. The method of claim 50, wherein the reference biological sample is a non-diseased sample.

52. The method of claim 50, wherein the reference biological sample is a diseased sample.

53. The method of claim 50, wherein the comparing step results in identifying biomarkers for the diseased sample.

54. (canceled)

55. A non-transitory computer-readable medium having stored thereon instructions that, when executed by at least one processor, cause the at least one processor to perform a method for profiling a target aptamer library for a target biological sample, the method comprising:
(i) acquiring the information of the target aptamer library for the target biological sample from a first aptamer database,
(ii) acquiring the information of a reference aptamer library for a reference biological sample from a second aptamer database, and
(iii) comparing the information of the target aptamer library for the target biological sample and the reference aptamer library for the reference biological sample thereby profiling the target aptamer library,
wherein the first aptamer database in step (i) is the aptamer database of claim 35.

56.-73. (canceled)
